(19) United States
(12) Patent Application Publication
(10) Pub. No.: US 2016/0334396 A1
(43) Pub. Date: Nov. 17, 2016

(54) BIOMARKER ASSAY USING MICROPARTICLE AGGREGATION

(71) Applicants: Gang CHENG, Fairlawn, OH (US); Jiang Zhe, Copley, OH (US); Yu Han, Akron, OH (US); HaiYan Wu, Broadview Heights, OH (US)

(72) Inventors: Gang CHENG, Fairlawn, OH (US); Jiang Zhe, Copley, OH (US); Yu Han, Akron, OH (US); HaiYan Wu, Broadview Heights, OH (US)

(73) Assignee: The University of Akron, Akron, OH (US)

(21) Appl. No.: 14/855,870

(22) Filed: Sep. 16, 2015

Related U.S. Application Data
(60) Provisional application No. 62/160,014, filed on May 12, 2015.

(51) Int. Cl. G01N 33/543; G01N 15/06; G01N 15/02

(52) U.S. Cl. CPC G01N 33/54326; G01N 15/0227; G01N 15/0656; G01N 2015/0092

(57) ABSTRACT

In various aspects and embodiments, the present invention is directed to versatile, label-free method for the quantitative and qualitative detection of biomarkers and/or other compounds in a fluid sample using functionalized microparticle aggregates. In these methods, micron-scale particles are functionalized to specifically interact with the biomarker being measured and added to the sample to form aggregates, the size and number of which are counted to find a volume fraction and/or number fraction of aggregates in the sample. There is a direct correlation between the volume fraction and number fraction of these aggregates and the concentration of the corresponding biomarker. By comparing the measured volume fraction and/or number fraction of aggregates in the sample to a calibration curve, the concentration of that biomarker may be determined even for biomarkers or other target compounds in samples at very low concentrations, without the need for fluorescence and enzyme labelling of antibodies.

### CROSS REFERENCE TO RELATED APPLICATIONS

This application claims the benefit of U.S. provisional patent application Ser. No. 62/160,014 entitled “Label-free Biomarker Assay in a Micro Resistive Pulse Sensor via Immunoaggregation,” filed May 12, 2015, and incorporated herein by reference in its entirety.

### REFERENCE TO GOVERNMENT SUPPORT

The invention was developed at least in part with the support of National Science Foundation grant number CMMI-1129727. The government may have certain rights in the invention.

### FIELD OF THE INVENTION

One or more embodiments of the present invention relates to a method for detecting and/or quantifying biomarkers, other compounds or microoganisms in a fluid. In certain embodiments, the present invention relates to methods for measuring the concentration of one or more biomarkers and/or other target molecule using aggregation of functionalized microparticles.

### BACKGROUND OF THE INVENTION

Biomarker detection represents an important task for many scientific fields such as disease diagnosis, biodefense, environmental monitoring and biological research. Biomarkers are molecular or cellular indicators that are used to measure and evaluate biological states of the target subjects. Among various types of biomarkers, macromolecular biomarkers present in blood, such as antigens, antibodies and enzymes, are of particular interest because the presence of various diseases is directly linked to abnormal concentrations of specific biomarkers in blood plasma. Thus, the quantitative detection of biomarker(s) plays an important role in diagnosing many diseases, evaluating the extent of a disease, and monitoring the response to therapy. Additionally, complex samples, available in small amounts, have to be processed quickly, preferably near a patient's bedside, so that medical responses can be adjusted more rapidly to the patient's reaction. Therefore, it is of the utmost importance to be able to detect and quantify biomarkers rapidly at low concentrations, with portable, inexpensive devices.

Immunoassay is a prevalent method for biomarker detection due to its high specificity. However, conventional immunoassays such as enzyme-linked immunosorbent assay (ELISA), require labelling antibodies, long assay time, and bulky, complicated detection instruments. Recently microfluidic immunosensing devices for biomarkers have been developed with various detection methods, including optical (fluorescent, luminescent or colorimetric), electrochemical, surface plasmon resonance (SPR), quartz crystal microbalance (QCM) and capillary electrophoretic immunoassays (CEIA). Most of these methods require labelling of a detection probe or optical detection or the modifications of sensing surfaces, and typically employ bulky, expensive and complicated detection instruments. Recently, resistive pulse sensing has been used to provide a label-free method for immunoassays. However, these methods are usually preformed in nanoscale sensing channels, which is problematic because: (i) fabrication of nanoscale sensing channels has been found to be very difficult, requiring an expensive and complex nanofabrication facility; and (ii) nanoscale devices have a very low throughput, i.e. each nanoscale channel can only handle a very small amount of sample at a time.

None of these methods are of much use for laboratories and clinics lacking immediate access of analytical instruments. Accordingly, what is needed in the art is a fast, highly sensitive and low cost immunoassay method for biomarkers and/or other target compounds, which does not require complex sample preparations or complex detection instrumentation and is compatible with commonly used analytical lab instruments.

### SUMMARY OF THE INVENTION

In various aspects and embodiments, the present invention is directed to sensitive, cost effective, versatile, and label-free method for the quantitative and qualitative detection of biomarkers and/or other compounds in a fluid sample using functionalized microparticle aggregates. In some embodiments, the present invention is directed to biomarker detection. In some embodiments, a resistive pulse sensor may be used to detect aggregates and measure the number and volume fractions of the aggregates. The lower detection limit is comparable with commercial available human ferritin ELISA kits (˜0.1 ng/ml) and can be further extended using lower concentrations of functionalized microparticles and higher affinity capture ligands. It has been demonstrated that the detection range of the biomarker or other target molecules can be accurately tuned by changing the functionalized microparticle concentration. This method can be readily adapted for the detection and quantification of any biomacromolecules or other compounds as long as there are high affinity capture probes available. Furthermore, these capture ligands/probes are not limited to antibodies. In comparison to conventional sandwich ELISA method, the methods of the various embodiments of the present invention enable cost-effective, fast biomarker detection with high sensitivity, and requires no complex measurement setup and sample preparations

In one or more embodiments, the present invention is directed to a method for measuring the concentration of a compound in a fluid comprising: preparing a fluid sample containing an unknown concentration of a compound to be measured; preparing a plurality of functionalized microparticles, the plurality of functionalized microparticles being functionalized to specifically interact with the compound; combing the plurality of functionalized microparticles with the fluid sample containing an unknown concentration of the compound, wherein the interaction between the functionalized microparticles and the compound is sufficient to cause the functionalized microparticles to aggregate around the compounds in the sample, thereby forming compound-microparticle aggregates; counting the number and size of the compound-microparticle aggregates; calculating a volume fraction or number fraction of the compound-microparticle aggregates in the sample, based upon the number and size of the compound-microparticle aggregates in the sample; preparing a calibration curve comprising the volume fraction or number fraction of compound-microparticle aggregates formed at known concentrations of the compound and the functionalized microparticles; and comparing the volume fraction of the compound-microparticle aggregates found in the counting and calculating step to the calibration curve to find the concentration of the compound in the fluid sample.

In some embodiments, the plurality of functionalized microparticles have a diameter of from 0.5 μm or more to 10 μm or less. In some embodiments, the plurality of functionalized microparticles have a diameter of from 0.2 μm or more to 0.5 μm or less. In some embodiments, the plurality of functionalized microparticles have a diameter of from 0.5 μm or more to 5 μm or less. In some embodiments, wherein the plurality of functionalized microparticles have a diameter of from 5 μm or more to 10 μm or less. In some embodiments, wherein the plurality of functionalized microparticles have a diameter of from 10 μm or more to 50 μm or less.

In some embodiments, the method for measuring the concentration of a compound in a fluid may comprise one or more of the above embodiments, wherein the plurality of functionalized microparticles are magnetic. In some embodiments, the method for measuring the concentration of a compound in a fluid may comprise one or more of the above embodiments, wherein the functionalized microparticles further comprise a fluorescent molecule. In some embodiments, the method for measuring the concentration of a compound in a fluid may comprise one or more of the above embodiments, wherein the plurality of functionalized microparticles comprise polystyrene, latex, gold, silica, organic materials, inorganic materials or combinations thereof. In some embodiments, the method for measuring the concentration of a compound in a fluid may comprise one or more of the above embodiments, wherein the plurality of functionalized microparticles are functionalized with one or more capture ligands selected from the group comprising antibodies, proteins, peptides, nucleic acids, aptamers, poly/oligo/mono saccharides, and combinations thereof.

In some embodiments, the method for measuring the concentration of a compound in a fluid may comprise one or more of the above embodiments, wherein the compound-microparticle aggregates comprise one compound and at least two functionalized microparticles. In some embodiments, the method for measuring the concentration of a compound in a fluid may comprise one or more of the above embodiments, wherein the compound to be measured is selected from the group consisting of ferritin, alanine transaminase (ALT), aspartate transaminase (AST), anti-hCG antibody, carcinoembryonic antigen (CEA), Alpha-Fetoprotein (AFP), AFP-L3, prostate specific antigen (PSA), C-reactive protein (CRP), estrogen receptor/progesteron receptor, receptor tyrosine-protein kinase erbB-2, (HER-2/neu), the epidermal growth factor receptor (EGFR), V-Ki-ras2 Kirsten rat sarcoma viral oncogene homolog (KRAS), UDP glucuronosyltransferase 1 family (UGT1A1), receptor tyrosine kinase (c-KIT), CD20 Antigen, CD30, fip1-like-1 fused with platelet derived growth factor receptor alpha (FIP1L1-PDGRFalpha), Platelet-derived growth factor receptors (PDGFR), Philadelphia Chromosome (BCR/ABL), PML/RAR alpha, thiopurine S-methyltransferase (TPMT), anaplastic lymphoma kinase (ALK), V-Ki-ras2 Kirsten rat sarcoma viral oncogene homolog (KRAS), serine/threonine-protein kinase B-Raf (BRAF), peptides, poly/oligo-saccharide, nucleic acids, lipoproteins, other biomolecules, virus, microplasma, bacteria, and combinations thereof. In some embodiments, the method for measuring the concentration of a compound in a fluid may comprise one or more of the above embodiments, wherein the concentration of the compound in the sample is from about 1 pg/mL or more to about 100 mg/mL or less.

In some embodiments, the method for measuring the concentration of a compound in a fluid may comprise one or more of the above embodiments, wherein the step of counting the number and size of the compound-microparticle aggregates performed using a resistive pulse sensor, an optical microscope, a fluorescence microscope, a flow cytometer, or a particle counter. In some embodiments, the method for measuring the concentration of a compound in a fluid may comprise one or more of the above embodiments, wherein the step of counting the number and size of the compound-microparticle aggregates is performed using a resistive pulse sensor. In some embodiments, the method for measuring the concentration of a compound in a fluid may comprise one or more of the above embodiments, wherein the resistive pulse sensor further comprises a channel having an area, the plurality of functionalized microparticles has a projected area, and the projected area of one of the plurality of microparticles is from about 1% or more to about 50% or less of the area of the channel. In some embodiments, the method for measuring the concentration of a compound in a fluid may comprise one or more of the above embodiments, wherein the resistive pulse sensor has two or more channels for counting the number and size of the compound-microparticle aggregates.

In some embodiments, the method may comprise one or more of the above embodiments, wherein the fluid sample contains an unknown concentration of two or more different compounds to be measured; the step of preparing a plurality of functionalized microparticles further comprises preparing a plurality of functionalized microparticles for each one of the two or more different compounds to be measured; the step of combining further comprises forming a compound-microparticle aggregate for each of the compounds being measured; the step of counting further comprises placing the sample containing compound-microparticle aggregates for each of the compounds being measured in a multichannel resistive pulse sensor, the multichannel resistive pulse sensor simultaneously measuring the number and size of the compound-microparticle aggregates for each of the compounds to be measured; the step of calculating further comprising the step of calculating the volume fraction or number fraction of the compound-microparticle aggregates for each compound to be measured in the sample; the step of preparing a calibration curve further comprises preparing a calibration curve for each for each compound to be measured in the fluid sample; and the step of comparing further comprises comparing the volume fraction of each of compound-microparticle aggregates found in the counting and calculating steps to its corresponding calibration curve to find the concentration of each compound to be measured in the fluid sample.

In some embodiments, the method may comprise one or more of the above embodiments, wherein the fluid sample contains an unknown concentration of two or more different compounds to be measured; the step of preparing a plurality of functionalized microparticles further comprises preparing functionalized microparticles of a different size for each one of the two or more different compounds to be measured; the step of combining further comprises forming a compounds-microparticle aggregate for each of the compounds being measured; the step of counting further comprises placing the sample containing compound-microparticle aggregates for each of the compounds being measured in a resistive pulse sensor, multichannel resistive pulse sensor, or particle counter, the resistive pulse sensor, multichannel resistive pulse sensor, or particle counter simultaneously measuring the number and size of the compound-microparticle aggregates for each of the compounds to be measured; the step of calculating further comprising the step of calculating the volume fraction or number fraction of the compound-microparticle aggregates for each compound to be measured in the sample; the step of preparing a calibration curve further comprises preparing a calibration curve for each for each compound to be measured in the sample; and the step of comparing further comprises comparing the volume fraction or number fraction of each of compound-microparticle aggregates found in the counting and calculating steps to its corresponding calibration curve to find the concentration of each compound to be measured in the fluid sample.

In some embodiments, the method may comprise one or more of the above embodiments, wherein the fluid sample contains an unknown concentration of two or more different compounds to be measured; the step of preparing a plurality of microparticles further comprises preparing a plurality of functionalized microparticles for each of the two or more different compounds to be measured, the functionalized microparticles for each of the two or more different compounds to be measured having a different color; the step of combining further comprises forming a compounds-microparticle aggregate for each of the compounds being measured; the step of counting further comprising placing the sample containing the compound-microparticle aggregates for each of the compounds being measured in an optical microscope and measuring the number and of the compound-microparticle aggregates for each of the colors; the step of calculating further comprising the step of calculating the number fraction of the compound-microparticle aggregates present for each compound to be measured in the sample; the step of preparing a calibration curve further comprises preparing a calibration curve for each compound to be measured in the sample; and the step of comparing further comprises comparing the number fraction of each of compound-microparticle aggregates found in the calculating step to its corresponding calibration curve to find the concentration of each compound to be measured in the fluid sample.

In some embodiments, the method may comprise one or more of the above embodiments, wherein the fluid sample contains an unknown concentration of two or more different compounds to be measured; the step of preparing a plurality of functionalized microparticles further comprises preparing a plurality functionalized microparticles for each of the two or more different compounds to be measured, wherein the functionalized microparticles for each of the two or more different compounds to be measured have a different fluorescence spectrum; the step of combining further comprises forming a compound-microparticle aggregates for each of the compounds being measured; the step of counting further comprises placing the sample containing the compound-microparticle aggregates for each of the compounds being measured in a fluorescent microscope or flow cytometer, the optical microscope measuring the number of the compound-microparticle aggregates and the flow cytometer measuring the number and size of the compound-microparticle aggregates at the fluorescence spectrum for each one of the compounds to be measured; the step of calculating further comprising the step of calculating the volume or number fraction of the compound-microparticle aggregates for each compound to be measured in the sample; the step of preparing a calibration curve further comprises preparing a calibration curve for each for each compound to be measured in the sample; and the step of comparing further comprises comparing the volume or number fraction of each of the compound-microparticle aggregates found in the counting and calculating steps to its corresponding reference curve to find the concentration of each compound to be measured in the fluid sample.

### BRIEF DESCRIPTION OF THE DRAWINGS

For a more complete understanding of the features and advantages of the present invention, reference is now made to the detailed description of the invention along with the accompanying figures in which:

FIG. 1 is a schematic showing a biomarker assay mechanism according to one or more embodiments of the present invention. Target compounds and functionalized microparticles form compound-MP aggregates, which are detected by a microfluidic resistive pulse sensor.

FIG. 2 is a schematic of a microscale resistive pulse sensor, including insets showing microscopy images of the on-chip filter and sensing channel.

FIG. 3 is a graph showing relative resistive pulses caused by single functionalized microparticles (MPs), two functionalized microparticle-compound aggregates, (2-MP) and three functionalized microparticle-compound aggregates (3-MP). The biomarker compound (ferritin) and MP concentrations were 41.6 ng/mL and 53.40 μg/mL.

FIG. 4 is a graph showing counts and size distribution of ferritin-MP aggregates according to one or more embodiments of the present invention. The inset is an optical image of single MPs and formed 2-MP and 3-MP aggregates. Ferritin and MP concentrations were 41.6 ng/mL and 53.40 μg/mL. The total particle count was 4.9×10⁴ formed by approximately 6.4×10⁴ MPs in 20 μL of sample.

FIG. 5 is a schematic illustration of the principle of an immunoaggregation assay according to one or more embodiments of the present invention, which can be readily coupled with optical microscopes or particle counters for quantitative and qualitative detection of biomacromolecules and other target compounds.

FIG. 6 is a graph showing the volume fraction of ferritin-MP aggregates prepared according to one or more embodiments of the present invention at different concentrations of ferritin in 10% FBS solution. Three sets of MP concentrations were used: 13.35 μg/mL (lines with circles), 53.40 (lines with triangles), and 213.40 μg/mL (lines with squares).

FIG. 7 is a graph showing the volume fraction of ferritin-MP aggregates prepared according to one or more embodiments of the present invention at different concentrations of ferritin in PBS (lines with circles) and the corresponding logistic function fill (solid line). The concentration of MP was 53.40 μg/mL for all tests. Ten samples were prepared and measured at each ferritin concentration.

FIG. 8 is a graph showing the number fraction of rAb-MP aggregates to all particles as a function of goat IgG concentration in PBS containing 0.1% BSA. Particle counts were obtained from optical bright field microscope images. The standard deviation was calculated from three replicates.

FIG. 9 is a graph showing the volume fraction of gAb-MP aggregates to all particles as a function of human ferritin concentration in PBS containing 0.1% BSA. Particle counts were obtained from Accusizer. The standard deviation was calculated from five replicates.

FIG. 10 is a graph showing the number fraction of gAb-MP aggregates to all particles as a function of human ferritin concentration in 10% FBS at 53.40 μg/mL (dashed line with diamonds) and 213.40 μg/mL (solid line with circles) of gAb-MP. Particle counts were obtained from bright field microscope images. The standard deviation was calculated from three replicates.

FIG. 11 is a schematic representation of a multiplexed multichannel resistive pulse sensor.

FIG. 12A-B are Fluorescence Microscope images showing: (FIG. 12A) FITC labeled rAb-MP without goat Ig G and (FIG. 12B) FITC labeled rAb-MP with 36 ng/mL goat IgG as a model biomarker. The concentration of rAb-MP was kept constant at 53.4 μg/mL.

FIG. 13A-B are graphs showing accusizer measurement results for (FIG. 13A) FITC labeled rAb-MP without goat Ig G and (FIG. 13B) FITC labeled rAb-MP with 36 ng/mL goat IgG as a model macromolecular biomarker. The concentration of rAb-MP was kept constant at 53.4 μg/mL.

FIG. 14 is a schematic representation of the multiplexed aggregation assay mechanism and design concept for a two-stage resistive pulse sensor (RPS) using the magnetic properties of the functionalized microparticles.

FIG. 15 is a graph showing typical counts and size distributions of two types of Ab-MPs and their aggregates. The inset is a microscopy image of anti-mouse MPs (2.0 μm), anti-ferritin MPs (2.8 μm) and their doublets. The mouse anti-rabbit IgG concentration was 24.0 ng/mL, human ferritin concentration was 208 ng/mL. The mixture of Ab-MPs probes consist of 4.7×103 count/μL of anti-mouse and 1.4×104 count/μL of anti-ferritin MPs.

FIG. 16 is a graph showing the correlation between the concentration of mouse anti-rabbit IgG ranging from 3.1 to 51.2×103 ng/mL and volume fraction of anti-ferritin MPs doublet (f).

FIG. 17 is a graph showing the correlation between the concentration of human ferritin ranging from 5.2 to 208 ng/mL and volume fraction of anti-ferritin MPs doublet (f₂). The volume ratio of non-specific anti-mouse MPs doublet were 5.3±0.5%.

FIG. 18 is a graph showing counts and size distribution of two types of Ab-MPs and their aggregates measured by the two-stage RPS.

FIG. 19 is a graph comparing the volume fraction of the non-magnetic MPs doublets measured by 1st stage and 2nd stage RPS, respectively.

FIG. 20 is a schematic diagram showing a fabrication process for the two-layer SU8 mold and the resistive pulse sensor device

FIG. 21A-C are graphs regarding calibration of the resistive pulse sensor. (FIG. 16A) is a graph showing 2.80 μm and 5.00 μm microparticles were used in the sizing calibration. The measured sizes were 2.80±0.16 μm and 4.91±0.37 μm. FIG. 21B is a graph showing the four concentrations of 2.80 μm microparticles that were used in the concentration calibration: 500 p/μl, 1000 p/μl, 2000 p/μl and 4000 p/μl. FIG. 21C is a graph showing the four concentrations of 2.80 μm microparticles that were used in the concentration calibration: 500 p/μl, 1000 p/μl, 2000 p/μl and 4000 p/μl, further showing the particle concentration of 875 p/μl (13.35 μg/ml) (circle) and 3500 p/μl (53.40 μg/ml)(triangle).

FIG. 22 is an electrical diagram of a representative electrical circuit for resistive pulse sensing.

### DETAILED DESCRIPTION OF THE ILLUSTRATIVE EMBODIMENTS

In various aspects and embodiments, the present invention is directed to versatile label-free method for the quantitative and qualitative detection of biomarkers and/or other target compounds in a fluid sample using functionalized microparticle aggregates. In these methods, generally micron-scale particles are functionalized to specifically interact with the biomarker or other compound being measured. When these functionalized particles are added to the sample, they interact with the corresponding biomarker (or other targeted compound) in the fluid sample to form aggregates, the size and number of which may be counted to find the volume fraction and/or number fraction of the aggregates in the sample. It has been found that at a given set of reaction conditions, there is a direct correlation between the volume fraction and/or number fraction of these aggregates and the concentration of the biomarker or other compound being measured. That is, within a calibration range, the higher the volume fraction and/or number fraction of aggregates, the higher the concentration of the biomarker or other compound being measured. Accordingly, in various embodiments of the present invention a calibration or reference curve may be prepared for a particular biomarker or other target compound being tested showing the volume fraction and/or number fraction of aggregates found using reference solutions having known concentrations of the biomarker or other target compound being tested. The concentration of that biomarker or other target compound in the sample can then be found by comparing the measured volume fraction and/or number fraction of these aggregates in the sample to the calibration curve. The methods of various embodiments of the present invention enable reliable detection of target macromolecular biomarkers or other target compounds in samples at very low concentrations, without the need for fluorescence and enzyme labelling of antibodies.

In one aspect, the present invention is directed to sensitive, versatile and cost effective methods for the quantitative and qualitative detection of a biomarker or other compound in a fluid. In some embodiments, the present invention is directed to methods of determining the concentration of a biomarker or other compound in a fluid. In one or more embodiments, the sensitive, versatile and cost effective methods for the quantitative and qualitative detection of a biomarker or other compound in a fluid comprise the step of preparing and/or obtaining a fluid sample containing an unknown concentration of a compound to be measured.

The fluids that may be used with the methods of the present invention are not particularly limited. It should be appreciated, however, that the fluid should be substantially free of any solid particulate material or particles that would interfere with the ability to count the number and size of the compound-microparticle aggregates being measured, and more broadly, should be compatible with the mechanism chosen for counting the number and size of the compound-microparticle aggregates being measured, as discussed below. In some embodiments, the fluid tested may be a biological fluid, environmental fluid, or any substances containing soluble target compounds. In some embodiments, the fluid tested may be blood or blood plasma. In some embodiments, the fluid tested may be urine. In some embodiments, the fluid tested may be lake water or river water for environmental monitoring.

While the present invention is often described herein with respect to finding the concentration of biomarkers, it is not so limited. In various embodiments, the methods of the present invention may be used to detect and/or quantify any compound for which a functionalized microparticle can be fabricated and may include, without limitation, ferritin, alanine transaminase (ALT), aspartate transaminase (AST), anti-hCG antibody, carcinoembryonic antigen (CEA), Alpha-Fetoprotein (AFP), AFP-L3, prostate specific antigen (PSA), C-reactive protein (CRP), estrogen receptor/progesteron receptor, receptor tyrosine-protein kinase erbB-2, (HER-2/neu), the epidermal growth factor receptor (EGFR), V-Ki-ras2 Kirsten rat sarcoma viral oncogene homolog (KRAS), UDP glucuronosyltransferase 1 family (UGT1A1), receptor tyrosine kinase (c-KIT), CD20 Antigen, CD30, fip1-like-1 fused with platelet derived growth factor receptor alpha (FIP1L1-PDGRFalpha), Platelet-derived growth factor receptors (PDGFR), Philadelphia Chromosome (BCR/ABL), PML/RAR alpha, thiopurine S-methyltransferase (TPMT), anaplastic lymphoma kinase (ALK), V-Ki-ras2 Kirsten rat sarcoma viral oncogene homolog (KRAS), serine/threonine-protein kinase B-Raf (BRAF), peptides, poly/oligo-saccharide, nucleic acids, lipoproteins, other biomolecules, virus, microplasma, bacteria, and/or combinations thereof.

In various embodiments, the methods of the present invention provide for the quantitative and qualitative detection of biomarkers and/or other compounds present in the sample fluid at very low concentrations. It should be appreciated that the sensitivity of the methods of the various embodiments of the present invention will depend upon the particulars of the method being used including the affinity of the capture ligand, the size of the target compound and the concentration of the microparticles. In some embodiments, the methods of the present invention can detect and quantify biomarkers and/or other compounds present in the sample in concentrations of from about 1 pg/mL or more to about 100 mg/mL or less. In some embodiments, the methods of the present invention can detect and quantify biomarkers and/or other compounds present in the sample in concentrations of from about 1 pg/mL or more to about 1 ng/mL or less. In some embodiments, the methods of the present invention can detect and quantify biomarkers and/or other compounds present in the sample in concentrations of from about 1 ng/mL or more to about 1 μg/mL or less. In some embodiments, the methods of the present invention can detect and quantify biomarkers and/or other compounds present in the sample in concentrations of from about 1 μg/mL or more to about 1 mg/mL or less. In some embodiments, the methods of the present invention can detect and quantify biomarkers and/or other compounds present in the sample in concentrations of from about 10 pg/mL or more to about 10 mg/mL or less. In some embodiments, the methods of the present invention can detect and quantify biomarkers and/or other compounds present in the sample in concentrations of from about 100 pg/mL or more to about 1 mg/mL or less. In some embodiments, the methods of the present invention can detect and quantify biomarkers and/or other compounds present in the sample in concentrations of from about 1 ng/mL or more to about 10 mg/mL or less. In some embodiments, the methods of the present invention can detect and quantify biomarkers and/or other compounds present in the sample in concentrations of from about 1 ng/mL or more to about 1 mg/mL or less. In some embodiments, the methods of the present invention can detect and quantify biomarkers and/or other compounds present in the sample in concentrations of from about 1 ng/mL or more to about 100 μg/mL or less. In some embodiments, the methods of the present invention can detect and quantify biomarkers and/or other compounds present in the sample in concentrations of from about 1 ng/mL or more to about 10 μg/mL or less.

In one or more embodiments, the sensitive, versatile and cost effective methods for the quantitative and qualitative detection of a biomarker and/or other compound in a fluid comprises the step of preparing a plurality of microparticles that are functionalized to specifically interact with the compound. In various embodiments, a wide variety of materials may be used to form the functionalized microparticles of the present invention. The functionalized microparticles of the present invention may be made from any material that is: (i) capable of forming micro-sized particles that do not dissolve or degrade quickly in the fluid to be tested, and (ii) capable of being functionalized with a plurality of capture ligands or other structures and/or materials that specifically interact with the compound being tested. Examples of materials that may be used as to form the microparticles may include, without limitation, polystyrene, latex, gold, silica, other organic and inorganic materials or combinations thereof. In some embodiments, the functionalized microparticles of the present invention may include commercially available streptavidin functionalized magnetic microparticles with an average diameter of 2.80 μm (Dynabeads M280, Life Technologies, USA).

As set forth above, the functionalized microparticles of the present invention are sized on a micrometer scale. It should be appreciated that the functionalized microparticles of the present invention should not be so small as to require fabrication and/or use of a nano-scale resistive pulse sensor having nanoscale sensing channels, require an expensive and complex nanofabrication facility, and/or have a very low throughput, or so large that steric hindrances prevent the formation of aggregates. In some embodiments, the functionalized microparticles of the present invention have a diameter of from 0.2 μm or more to 50 μm or less. In some embodiments, the functionalized microparticles of the present invention have a diameter of from 0.2 μm or more to 30 μm or less. In some embodiments, the functionalized microparticles of the present invention have a diameter of from 0.2 μm or more to 10 μm or less. In some embodiments, the functionalized microparticles of the present invention have a diameter of from 0.2 μm or more to 5 μm or less. In some embodiments, the functionalized microparticles of the present invention have a diameter of from 0.2 μm or more to 0.5 μm or less. In some embodiments, the functionalized microparticles of the present invention have a diameter of from 0.5 μm or more to 5 μm or less. In some embodiments, the functionalized microparticles of the present invention have a diameter of from 1.5 μm or more to 10 μm or less. In some embodiments, the functionalized microparticles of the present invention have a diameter of from 5 μm or more to 10 μm or less. In some embodiments, the functionalized microparticles of the present invention have a diameter of from 7 μm or more to 15 μm or less. In some embodiments, the functionalized microparticles of the present invention have a diameter of from 10 μm or more to 50 μm or less. In some embodiments, the functionalized microparticles of the present invention have a diameter of from 2.0 μm or more to 8 μm or less. In some embodiments, the functionalized microparticles of the present invention have a diameter of from 3 μm or more to 6 μm or less.

As set forth above, the functionalized microparticles various embodiments of the present invention are functionalized with a plurality of capture ligands that specifically interact with the compound being tested. The terms “specifically interact” and “specific interaction” are used herein interchangeably to refer to the ability of a material to interact with a target compound (e.g. the compound being tested) with higher affinity than nonspecific interaction using hydrogen bonding, van der waal interactions, and/or electrostatic interactions, and not to interact with other compounds in the sample. As used herein, the terms “capture ligand” and “capture probe” are interchangeable and refer to a compound, moiety, molecule, protein, nucleic acid, mono/oligo/polysaccharideor other material used to functionalize a microparticle that specifically interacts with a target compound. Examples of materials that may be used as capture ligands in one or more embodiment of the present invention may include, without limitation, antibodies, proteins, peptides, nucleic acids, aptamers, poly/oligo/mono saccharides, or any other suitable capture ligands known in the art. In some embodiments, the capture ligand may be one or more antibody for the target compound.

The specific mechanism for functionalizing the microparticles with one or more capture ligands will, of course, depend upon the specific microparticle and capture ligand involved, but any suitable method known in the art may be used. One of ordinary skill in the art will be able to functionalize microparticles with a desired capture ligand without undue experimentation. For example, capture ligands with amine groups can be functionalized to microparticles with carboxylate groups; capture ligand with biotin can be conjugated to microparticles with streptavidins, and antibody can be conjugated to microparticles with protein A and G.

In some embodiments, the microparticles to be functionalized may be magnetic. It has been found that magnetic microparticles are easy to handle during and after the functionalization process through the use of an external magnet. They can be washed away by removing external magnetic field; and captured by adding a magnetic field. In addition, use magnetic particle formed aggregates also enables detection of single or multiple biomarkers via measuring the aggregates' size and magnetic properties.

In some embodiments, the functionalized microparticles further comprise one or more fluorescent molecules, as described below.

In one or more embodiments, the sensitive, versatile and cost effective methods for the quantitative and qualitative detection of a biomarker or other compound in a fluid comprises the step of adding the functionalized microparticles described above to the fluid sample containing an unknown concentration of the compound being measured. Preferably, the functionalized microparticle/sample mixture is then stirred or agitated at room temperature for a period of from about 1 to about 60 mins, although this is not required. As set forth above, the capture ligands on the functionalized microparticles are designed to specifically interact with the compound being measured and as the functionalized microparticles and the compound come in contact, the interaction between functionalized microparticles and the compound is sufficient to cause the at least some portion of the functionalized microparticles to aggregate around some of the compounds in the sample, thereby forming compound-microparticle aggregates.

The degree to which functionalized microparticles and the compound being detected or quantified will form compound-microparticle aggregates will depend upon a variety of factors including, without limitation, the affinity of the capture ligands on the functionalized microparticles for the compound being detected or measured, the number of the capture ligands on the functionalized microparticles, the concentration of the functionalized microparticles in the mixture, the size of the functionalized microparticles in the mixture, the concentration of the compound being detected or measured in the sample; the duration and vigorousness with which the mixture is agitated or stirred, and the temperature.

The degree to which each of these variables will affect aggregation or, more broadly, the operation of the methods of the present invention will depend upon the particulars of the specific combinations and reaction conditions being used. By controlling the type of capture ligand selected and the number of capture ligands present, it is possible in some embodiments of the present invention to tune the affinity of the functionalized microparticles for the compound being detected or measured. As will be apparent, all other things being equal, an increase in the affinity and/or number of the capture ligands and/or the concentration of functionalized microparticles in the mixture, will correspond to an increase in the amount of aggregation within a certain range of compound concentration.

Similarly, modest increases in temperature and stirring/agitation will increase the number of interactions between the functionalized microparticles and the compound being detected or quantified, thereby increasing the amount of aggregation. However, it should also be appreciated that if the temperature is too high and/or the stirring/agitation too intense, the forming aggregates may be broken apart. In some embodiments, the functionalized microparticle/sample mixture is prepares at a temperature of from 0° C. or more to 90° C. or less. In some embodiments, the functionalized microparticle/sample mixture is prepares at a temperature of from 4° C. or more to 90° C. or less. In some embodiments, the functionalized microparticle/sample mixture is prepares at a temperature of from 4° C. or more to 15° C. or less. In some embodiments, the functionalized microparticle/sample mixture is prepares at a temperature of from 15° C. or more to 40° C. or less. In some embodiments, the functionalized microparticle/sample mixture is prepares at a temperature of from 40° C. or more to 70° C. or less. In some embodiments, the functionalized microparticle/sample mixture may be stirred or agitated for a period of from about 70° C. to about 90° C. In some embodiments, the functionalized microparticle/sample mixture may be stirred or agitated for a period of from about 1 s to about 120 min. In some embodiments, the functionalized microparticle/sample mixture may be stirred or agitated for a period of from about is to about 10 min. In some embodiments, the functionalized microparticle/sample mixture may be stirred or agitated for a period of from about 10 min to about 60 min. In some embodiments, the functionalized microparticle/sample mixture may be stirred or agitated for a period of from about 60 min to about 120 min.

A larger aggregation ratio will extend the detection range. Because the aggregation ratio increase with the increase of biomarker concentration. The max aggregation ratio is correlated to the upper detection limit of biomarker concentration. A larger ratio of aggregation will extend the biomarker detection range.

In some embodiments, compound-microparticle aggregates may comprise one molecule of the compound being detected or quantified and two or more functionalized microparticles. In some embodiments, compound-microparticle aggregates may comprise from about two to about ten functionalized microparticles. In some embodiments, compound-microparticle aggregates may comprise from about two to about three functionalized microparticles. In some embodiments, compound-microparticle aggregates may comprise from about two to about four functionalized microparticles. In some embodiments, compound-microparticle aggregates may comprise from about two to about five functionalized microparticles. In some embodiments, compound-microparticle aggregates may comprise from about two to about six functionalized microparticles. In some embodiments, compound-microparticle aggregates may comprise from about five to about ten functionalized microparticles. In some embodiments, compound-microparticle aggregates may comprise from about three to about seven functionalized microparticles.

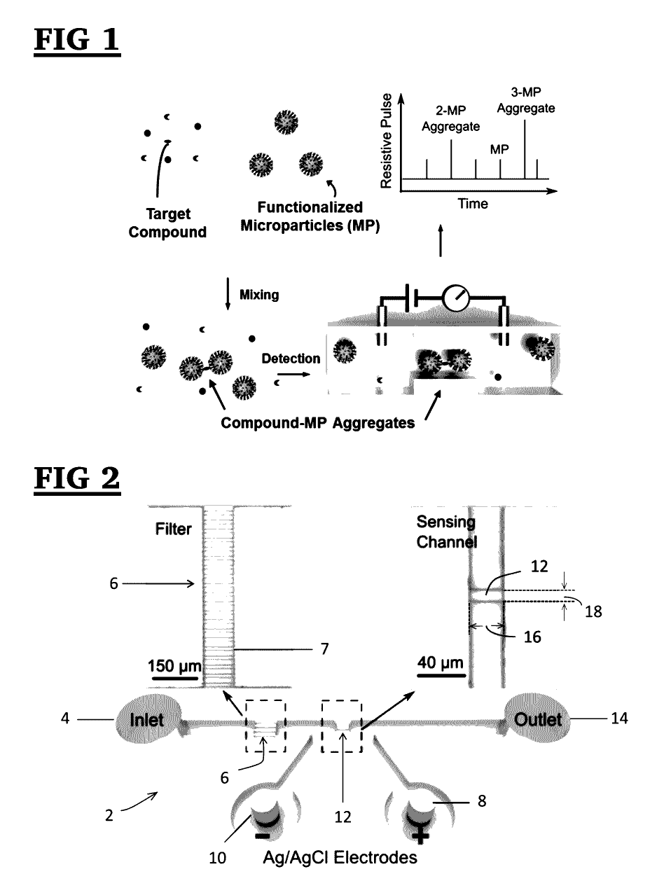

In the next step, the number and size of the functionalized microparticles and compound-microparticle aggregates are counted by any suitable means known in the art including, without limitation, a resistive pulse sensor, an optical microscope, a fluorescence microscope, a flow cytometer a particle counter or other device for counting and/or sizing particles. In some embodiments, the number and size of the functionalized microparticles and compound-microparticle aggregates may be counted as set forth in R. DeBlois and C. Bean, Review of Scientific Instruments, 2003, 41, 909-916, the disclosure of which is incorporated by reference herein in its entirety. FIG. 1 is a schematic diagram showing a representative method of performing various embodiments of the present invention using a resistive particle sensor. Resistive pulse sensors are known in the art and will be described herein only to the extent necessary to understand and practice the present invention. Any suitable resistive pulse sensor may be used to practice the present invention. In some embodiments, a suitable resistive pulse sensor may be constructed and tested as set forth in Example 1 below. A resistive pulse sensor according to one or more embodiment of the present invention is shown in FIG. 2. In the embodiment shown in FIG. 2, the resistive pulse sensor 2 has an inlet opening 4, an on-chip filter 6, a positive and negative electrode 8, 10, a sensing channel 12 and an outlet opening 14. In general operation, the sample being tested is placed in the inlet opening 4 using a micropipette or other suitable means and moves through filter 6 and sensing channel 12 to outlet opening 14 under pressure.

The pressure is preferably applied by orienting the resistive pulse sensor in such a way that the sample moves through the resistive pulse sensor using only the force of gravity, but the invention is not to be so limited. Any suitable method of forcing or drawing the sample from inlet opening 4 through filter 6 and sensing channel 12 to outlet opening 14, without allowing compound-microparticle aggregates in the sample fluid that has passed through sensing channel 12 to reenter sensing channel 12 and be counted twice.

In some embodiments, on-chip filter 6 having pores 7 is placed between inlet opening 4 and sensing channel 12 to filter out aggregates (and/or anything else), that are too large to pass through sensing channel 12 and be counted and sized. Accordingly, the size of the pores 7 in filter 6 will depend upon a variety of factors including the size of the functionalized microparticles and the size of the sensing channel 12 being used. In some embodiments, pores 7 on-chip filter 6 may have a width of 4.2 μm or more. In some embodiments, pores 7 on-chip filter 6 may have a width of from about 4.5 μm or more to about 10 μm or less. The approximate diameter of the functionalized microparticles may be estimated from a known diameter of the microparticles from which they were made or measured according to any one of a variety of ways known in the art for doing so. While it should be appreciated that the functionalized microparticles are not perfectly spherical, a projected cross sectional area of the functionalized microparticles may be calculated based upon an equivalent diameter of the functionalized microparticles being used. In some embodiments, the equivalent diameter may be calculated based on the resistive pulse sensor signal, which is associated with the projected area of the particle.

As can be seen in the enlargement of the sensing channel in FIG. 2, sensing channel 12, a width 18 and a height of 10 μm that define a cross sectional area. In some embodiments, sensing channel 12 has a length from about 2 μm to about 1000 μm. In some embodiments, sensing channel 12 has a length from about 2 μm to about 500 μm. In some embodiments, sensing channel 12 has a length from about 2 μm to about 300 μm. In some embodiments, sensing channel 12 has a length from about 2 μm to about 100 μm. In some embodiments, sensing channel 12 has a length from about 50 μm to about 1000 μm. In some embodiments, sensing channel 12 has a length from about 500 μm to about 1000 μm. In some embodiments, sensing channel 12 has a length from about 100 μm to about 700 μm. In some embodiments, sensing channel 12 has a length from about 150 μm to about 500 μm. In some embodiments, sensing channel 12 has a length equal to from about 1 time to about 50 times the diameter of the functionalized microparticles being used. In some embodiments, sensing channel 12 has a length equal to from about 1 time to about 25 times the diameter of the functionalized microparticles being used. In some embodiments, sensing channel 12 has a length equal to from about 1 time to about 10 times the diameter of the functionalized microparticles being used. In some embodiments, sensing channel 12 has a length equal to from about 10 times to about 50 times the diameter of the functionalized microparticles being used. In some embodiments, sensing channel 12 has a length equal to from about 30 times to about 50 times the diameter of the functionalized microparticles being used. In some embodiments, sensing channel 12 has a length equal to from about 5 time to about 25 times the diameter of the functionalized microparticles being used. In some embodiments, sensing channel 12 has a length equal to from about 10 times to about 20 times the diameter of the functionalized microparticles being used.

In some embodiments, sensing channel 12 will have a cross sectional area that is less than 100 times the projected area of the cross section of the functionalized microparticles to be used, but less than or equal to ten times the projected area of the cross section of the functionalized microparticles to be used. In some embodiments, sensing channel 12 will have a cross sectional area that is less than or equal to 50 times the projected area of the cross section of the functionalized microparticles to be used but less than or equal to 8 times the projected area of the cross section of the functionalized microparticles to be used. In some embodiments, sensing channel 12 will have a cross sectional area that is greater than or equal to 2 times the projected area of the cross section of the functionalized microparticles to be used but less than or equal to 6 times the projected area of the cross section of the functionalized microparticles to be used. In some embodiments, sensing channel 12 will have a cross sectional area that is greater than or equal to 2 times the projected area of the cross section of the functionalized microparticles to be used but less than or equal to 4 times the projected area of the cross section of the functionalized microparticles to be used. In some embodiments, sensing channel 12 will have a cross sectional area that is greater than or equal to 3 times the projected area of the cross section of the functionalized microparticles to be used but less than or equal to 10 times the projected area of the cross section of the functionalized microparticles to be used. In some embodiments, sensing channel 12 will have a cross sectional area that is greater than or equal to 5 times the projected area of the cross section of the functionalized microparticles to be used but less than or equal to 10 times the projected area of the cross section of the functionalized microparticles to be used. In some embodiments, sensing channel 12 will have a cross sectional area that is greater than or equal to 7 times the projected area of the cross section of the functionalized microparticles to be used but less than or equal to 10 times the projected area of the cross section of the functionalized microparticles to be used. In some embodiments, sensing channel 12 will have a cross sectional area that is greater than or equal to 3 times the projected area of the cross section of the functionalized microparticles to be used but less than or equal to 7 times the projected area of the cross section of the functionalized microparticles to be used.

Put another way, the projected cross sectional area of the functionalized microparticles being used will be from about 1% or more to about 50% or less of the cross sectional area of sensing channel 12. In some embodiments, the projected cross sectional area of the functionalized microparticles being used will be from about 5% or more to about 40% or less of the cross sectional area of sensing channel 12. In some embodiments, the projected cross sectional area of the functionalized microparticles being used will be from about 5% or more to about 30% or less of the cross sectional area of sensing channel 12. In some embodiments, the projected cross sectional area of the functionalized microparticles being used will be from about 5% or more to about 20% or less of the cross sectional area of sensing channel 12. In some embodiments, the projected cross sectional area of the functionalized microparticles being used will be from about 5% or more to about 10% or less of the cross sectional area of sensing channel 12. In some embodiments, the projected cross sectional area of the functionalized microparticles being used will be from about 15% or more to about 50% or less of the cross sectional area of sensing channel 12. In some embodiments, the projected cross sectional area of the functionalized microparticles being used will be from about 25% or more to about 50% or less of the cross sectional area of sensing channel 12. In some embodiments, the projected cross sectional area of the functionalized microparticles being used will be from about 35% or more to about 50% or less of the cross sectional area of sensing channel 12. In some embodiments, the projected cross sectional area of the functionalized microparticles being used will be from about 10% or more to about 40% or less of the cross sectional area of sensing channel 12. In some embodiments, the projected cross sectional area of the functionalized microparticles being used will be from about 15% or more to about 30% or less of the cross sectional area of sensing channel 12.

Turning back to FIG. 2, positive and negative electrodes 8, 10 are positioned on either side of sensing channel 12. When a voltage is applied across positive and negative electrodes 8, 10, a current is generated running from positive electrode 8 through sensing channel 12 and to negative electrode 10, with the material between positive and negative electrodes 8, 10 providing a resistance. As will be appreciated by those of skill in the art, when a functionalized microparticle or compound-microparticle aggregate enters sensing channel 12, it will cause an increase in the resistance (a resistance pulse) that is proportional to its size. In these embodiments, the number and magnitude of these pulses are recorded. The total number of these pulses indicates the total number functionalized microparticles and/or compound-microparticle aggregates passed through the sensing channel. The magnitude of these pulses indicates the relative size of particles in terms of the number of functionalized microparticles they contain.

In some embodiments, single functionalized microparticles (MP), compound-microparticle aggregates having two functionalized microparticles (2-MP), compound-microparticle aggregates having three functionalized microparticles (3-MP) etc. may be identified by the magnitude of the resistive pulses (δR/R), which is proportional to the particle volume (˜d³), using Equation 1:

δR/R=(d³/LD²)·[(D²/2L²)+1/\sqrt{1+(D/L)²}]·F(d³/D³) (Equation 1)

where R is the resistance of the sensing channel, d is the diameter of the functionalized microparticle, D and L are the characteristic diameter and the length of the rectangular sensing channel, F is the correction factor to be applied to the Maxwellian limit as a function of the ratio of particle to pore diameter on the basis of the upper limit theory. See R. DeBlois and C. Bean, Review of Scientific Instruments, 2003, 41, 909-916, the disclosure of which is incorporated by reference herein in its entirety. In some of these embodiments, D=(4·A/π)^{1/2}, where A is the cross-sectional area of the sensing channel. In some embodiments, F may be from about 1.0 or more to about 13.7 or less. In some embodiments, F may be 1.0. F is a numerical correction factor:

F(d³/D³)=1+1.264(d³/D³)+1.347(d³/D³)²+0.648(d³/D³)³+4.167(d³/D³)⁴

d is the equivalent diameter of the particle or aggregates.

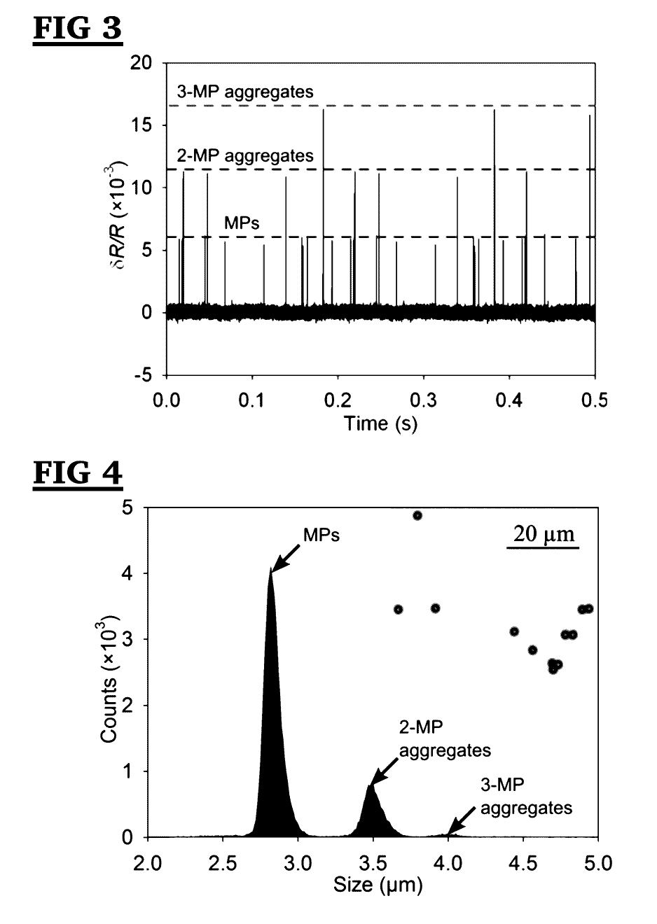

FIG. 3 shows the resistive pulses (δR/R) measured for a sample having functionalized microparticles (MP) and 2-MP and 3-MP sized compound-microparticle aggregates. As can be seen in FIG. 3, the magnitude of the resistive pulses (δR/R) measured is roughly proportional to the number of functionalized microparticles present. And as can be seen in FIG. 4, even though there is a range of sizes for the functionalized microparticles, 2-MP compound-microparticle aggregates, and 3-MP compound-microparticle aggregates, it is not difficult to distinguish between them.

In some embodiments, a multi-channel resistive pulse sensor may also be used. In these embodiments, the resistive pulse sensor will have two or more sensing channels arranged in parallel, each one having a set of electrodes an measuring changes in resistance as microparticles pass through. In some embodiments, described in more detail below, a multi-channel resistive pulse sensor may be used to detect and/or quantify multiple target compounds simultaneously.

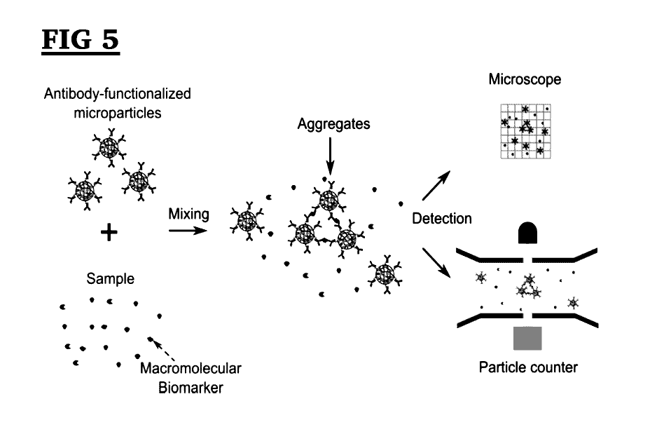

As set forth above, the methods counting the number and size of the present invention are not limited to the use of resistive pulse sensors. FIG. 5 is a schematic diagram showing a representative method of performing various embodiments of the present invention using a microscope or optical particle counter. In some other embodiments not pictured in FIG. 5, a fluorescence microscope or a flow cytometer may be used.

In some embodiments, a light microscope may be used to manually count the number and size of the compound-microparticle aggregates in a sample having a known volume. It should be appreciated that this method is very simple, relatively inexpensive, and does not require specialized equipment. It is, however, labor intensive, as all of the functionalized microparticles/compound-microparticle aggregates are counted manually. In these embodiments, the fluid sample being tested is preferably diluted so as to reduce the overall number of particles that must be counted.

In another similar embodiment, each of the functionalized microparticles may contain a fluorescent material/element. In these embodiments, a fluorescence microscope may be used to count the number and size of the functionalized microparticles/compound-microparticle aggregates. The fluorescent material/element included in the functionalized microparticles of these embodiments of the present invention is not particularly limited and may include, without limitation, fluorescent dyes, quantum dot, carbon dot, and/or fluorescent organic and inorganic particles. As should be apparent, the fluorescence of the functionalized microparticles/compound-microparticle aggregates when viewed on a fluorescence microscope make it easier for a technician to count the number and size of the functionalized microparticles/compound-microparticle aggregates in the sample, but also require the additional step of including the fluorescent material/element in the functionalized microparticles and access to a more expensive fluorescence microscope, adding to the expense and complexity of the operation. As with the light microscope described above, in these embodiments, the fluid sample being tested is preferably diluted so as to reduce the overall number of particles that must be counted.

In some embodiments, the device responsible for counting the number and size of the functionalized microparticles and compound-microparticle aggregates in the sample may comprise or be connected to a microprocessor and/or computer. One of ordinary skill in the art will be able to program a microprocessor and/or computer to collect the resistive pulse data and to calculate the number and size of the functionalized microparticles/compound-microparticle aggregates in the sample without undue experimentation. In some embodiments, Laboratory Virtual Instrument Engineering Workbench (LabVIEW™) (National Instruments Corporation, Austin, Tex.) may be used to collect the resistive pulse data and matrix laboratory (MATLAB™)(The MathWorks, Inc, Natick, Mass.) may be used to calculate the number and size of the functionalized microparticles/compound-microparticle aggregates in the sample.

As shown in FIG. 5 above, in some other embodiments of the present invention, an optical or electrical particle counter may be used to count the number of functionalized microparticles/compound-microparticle aggregates in the sample. Suitable optical or electrical particle counters are well known in the art and may include, without limitation, accusizer, coulter counter, or flow cytometer. In some other embodiments of the present invention, a flow cytometer may be used to count the number and size of functionalized microparticles/compound-microparticle aggregates in the sample.

Once the number and/or size of the functionalized microparticles/compound-microparticle aggregates in the sample tested is known, they may be used to calculate the volume fraction and/or number fraction of the compound-microparticle aggregates in the sample. One of ordinary skill in the art will be able to calculate the volume fraction and number fraction of compound-microparticle aggregates in the sample from the measured number and/or size functionalized microparticles/compound-microparticle aggregates in the sample without undue experimentation. In some embodiments, the volume fraction of compound-microparticle aggregates in a sample may be calculated by dividing the total volume of compound-microparticle aggregates by the total volume of the functionalized microparticles and compound-microparticle aggregates. In embodiments where there are 2-MP, 3-MP and 4-MP level compound-microparticle aggregates, for example, the volume fraction may be calculated using the formula:

$$Volume fraction = \frac{{n}_{2} \frac{4}{3} π {r}_{2}^{3} + {n}_{3} \frac{4}{3} π {r}_{3}^{3} + {n}_{4} \frac{4}{3} π {r}_{4}^{3}}{{n}_{1} \frac{4}{3} π {r}_{1}^{3} + {n}_{2} \frac{4}{3} π {r}_{2}^{3} + {n}_{3} \frac{4}{3} π {r}_{3}^{3} + {n}_{4} \frac{4}{3} π {r}_{4}^{3}} \qquad ( Equation 2 )$$

wherein n₁ is the number and r₁ is the radius of the functionalized microparticles in the sample; and n₂, n₃, n₄ and r₂, r₃, r₄ are, respectively, the number and radius of 2-MP, 3-MP and 4-MP level compound-microparticle aggregates in the sample.

Similarly, in some embodiments, the number fraction of compound-microparticle aggregates in a sample may be calculated by dividing the total number of compound-microparticle aggregates by the total number of the functionalized microparticles and compound-microparticle aggregates. In embodiments where there are 2-MP, 3-MP and 4-MP level compound-microparticle aggregates, for example, the number fraction may be calculated using the formula:

$$Number fraction = \frac{{\sum }_{i = 1}^{k} {n}_{i}}{{\sum }_{i = 1}^{k} {n}_{i}} \qquad ( Equation 3 )$$

wherein k is the number of the aggregates of 2-MP, 3-MP, 4-MP to k-MP level compound-microparticle aggregates from i to infinity; i is the starting aggregation level; and nᵢ is the number of aggregates at the aggregation level. Since the number of aggregates formed by more than three functionalized microparticles are very small and often negligible, in some embodiments, it is assumed that k=3 and, in these embodiments, the number fraction may be calculated using the formulas:

$$\begin{matrix}Number fraction = \frac{2 * {n}_{2} + 3 * {n}_{3}}{{n}_{1} + 2 * {n}_{2} + 3 * {n}_{3}} \\ or & ( Equation 4 ) \\ Number fraction = \frac{{n}_{2 +} {n}_{3 +} {n}_{4}}{{n}_{1 +} {n}_{2 +} {n}_{3 +} {n}_{4}} & ( Equation 5 )\end{matrix}$$

wherein n₁ is the number of the mono-functionalized microparticles in the sample and n₂, n₃, and n₄ are, respectively, the number for 2-MP, 3-MP and 4-MP level compound-microparticle aggregates.

As set forth above, it has been found that at a given concentration of functionalized microparticles, the number or volume ratio of compound-microparticle aggregates to total number of microparticles in the sample (functionalized microparticles and compound-microparticle aggregates) is proportional to the concentration of the biomarker or other target compound. This relationship provides the ability to quantify the concentration of the biomarker or other target compound by comparing the measured volume or number ratios for the biomarker or other target compound, to a calibration curve made using known concentrations of the biomarker or other target compound at the same reaction conditions and the same concentration of functionalized microparticles used to generate the measured volume or number ratios for the biomarker or other target compound. It should also be appreciated that this relationship holds true provided only that the reaction conditions and concentration of functionalized microparticles used for the calibration curve and for the actual measurement are essentially the same.

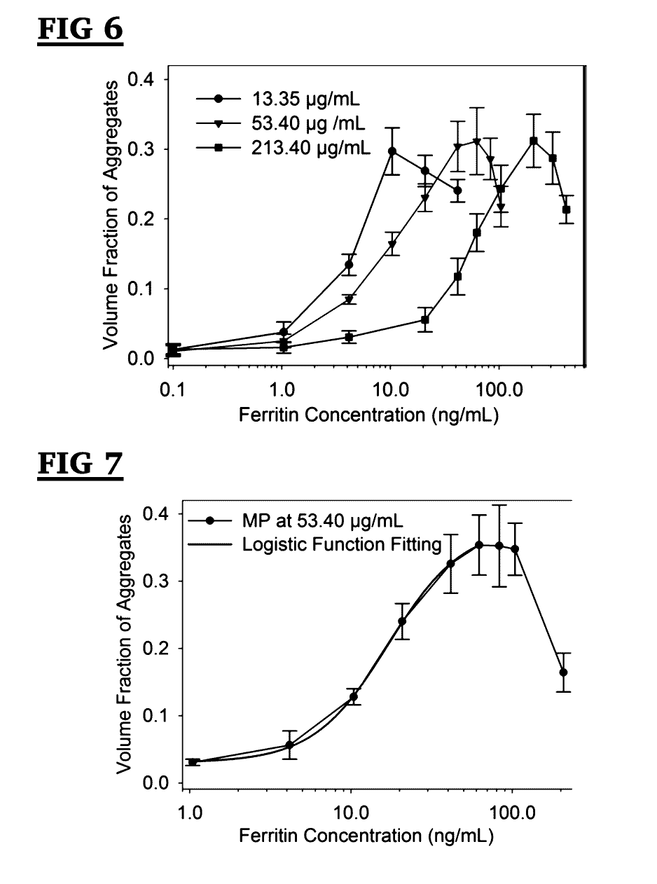

FIGS. 6-10 are examples of calibration curves. As can be seen, these calibration curves outline the effective detection range for the methods of the present invention. Since biomarkers and/or other target molecules exist at different concentrations, it is highly desired that the detection range of the assay be adjustable to match the various concentrations of target molecules. FIG. 6 shows that another significant advantage of the detection methods of various embodiments of the present invention is that the detection range can be tuned by adjusting the concentration of functionalized microparticles. As shown in FIG. 6, by decreasing and increasing the concentrations of functionalized microparticles, the lower and upper detection limits can be extended. Although higher functionalized microparticle concentrations provide a larger detection range, a lower functionalized microparticle concentration is more sensitive to lower biomarker concentration. Hence, for a biomarker or other target compound concentration exceeding the upper detection range, the correct target compound concentration can be determined by adding a test with increased microparticle concentration.

Alternatively, in some embodiments where target compound concentration in the sample exceeds the upper detection range, the sample may be diluted in series until the concentration of the target compound is within the detection range and the concentration of the starting sample calculated from the measured concentration of the target compound in the diluted sample. As the reaction conditions and concentration of functionalized microparticles are simple and easily reproducible, it is envisioned that detailed sets of calibration curves may be produced and standardized for various types and concentrations of functionalized microparticles.

To further decrease the lower detectable biomarker concentration, one possible approach is to use a capture ligand with a higher binding affinity to the given biomarker, which is expected to improve the volume fraction at lower biomarker concentrations. Since the binding of capture ligands to the target compounds is a reversible and concentration-dependent process, the capture ligands with the higher binding affinity are able to capture the target compounds and subsequently cause the aggregation at a lower concentration. Under flow conditions, a stronger interaction between the compound and the capture probe can reduce the disassociation of aggregates caused by the shear stress in the microfluidic channel and subsequently increase the detection sensitivity.

Further, as will be appreciated by those of skill in the art, nonspecific binding of non-target biomolecules to capture probes or sensing surfaces has been a long standing challenging issue for nearly all biomarker detection in complex media, such as blood, urine, body fluid, etc. and this nonspecific binding may cause the false positive result and decrease the detection sensitivity. However, the functionalized microparticles of various embodiments of the present invention have been found to be stable in the complex media and nonspecific binding of non-target protein has no effect on the aggregation of functionalized microparticles. FIG. 6, for example, shows a clear relationship between the biomarker concentration and the volume fraction of aggregates. As the aggregation-based detection methods of embodiments of the present invention are generally insensitive to other biomolecules present in a complex media, it is believed that these methods can be used for biomarker detection in complex media, i.e. human blood.

Last, the number or volume fraction of the compound-microparticle aggregates may be compared to the appropriate calibration curve to find the concentration of the target compound in the fluid sample. It should be noted, however, that at the upper end of the calibration curves prepared according to the methods of the present invention, a volume fraction or number fraction may have more than one corresponding concentration. (See, FIGS. 6-10). While not wishing to be bound by theory, it is believed that this may be caused by the aggregation behavior of the compound and functionalized microparticles. Accordingly, in some embodiments, it may be necessary to do a second or third assay with a diluted sample to confirm the accuracy of the first result. The second and the third assay can be used to confirm that the first assay fell in the detection range of the standard curve.

Also, while quantitative detection of biomarkers and or other target compounds is needed for many applications as set forth above, it should be appreciated that some applications call only for relatively quick qualitative detection of biomarkers and/or other target compounds. In these embodiments, a threshold number of compound-microparticle aggregates in a fixed volume can be predetermined given a fixed sample volume and functionalized microparticle concentration. In some embodiments, for example, an assay may be made to determine whether a sample contains a minimum concentration or a target compound. In these embodiments, the predetermined value may the number of aggregates that corresponds to the minimum concentration or a target compound, given a fixed sample volume and functionalized microparticle concentration. In some other embodiments, for example, an assay may be made to determine a target compound is present in the sample at any concentration. In these embodiments, the predetermined value may be any aggregation at all. In any case, sample can be considered as the positive once the number of aggregates measured exceeds the predetermined threshold value.

In another aspect, the present invention is directed to sensitive, versatile and cost effective methods for the quantitative and qualitative detection of two or more biomarkers and/or other compounds in a fluid. In some of these embodiments, a multiplexed multichannel resistive pulse sensor can be used to further reduce the detection time and detect multiple biomarkers in parallel. A representative example of a multiplexed multichannel resistive pulse sensor 20 is shown in FIG. 11. In the embodiment shown in FIG. 11, multichannel resistive pulse sensor 20 has an sample input 22 which is divided into four channels 24, 26, 28, 30. In these embodiments, different functionalized microparticles as described above are prepared for each one of the two or more different target compounds to be measured, in the manner set forth above. Each one of these is added to one of channels 24, 26, 28, 30 through corresponding functionalized microparticle inputs 32, 34, 36, 38 and are mixed in corresponding micromixers 40, 42, 44, 46. When these functionalized microparticles are added to the sample as described above, a different type of compound-microparticle aggregate is formed for each of the compounds being measured. Once formed compound-microparticle aggregates before passing through corresponding sensing channels 48, 50, 52, 54, and out through outputs 56, 58, 60, 62

In some of these embodiments, the number and size data for each type of target compound is multiplexed and sent to a single set of detection and processing equipment, usually a microprocessor or computer. As set forth above, each channel carries one type of microparticles in the inlet reservoir and is able to detect one type biomarker. In these embodiments each channel is encoded with a unique AC frequency (f₁, f₂, f₃ f₄). In these embodiments, the combined signal from all channels, containing multiple unique frequencies will be detected by only one set of measurement electronics. The combined signal can be decoded to obtain individual signals for each channel using fast Fourier Transform. In some other embodiments, each channel has its own set of measurement electronics.

In these embodiments, separate calibration curves are prepared for each for each target compound and its corresponding functionalized microparticle and the measured volume fraction or number fraction of each of compound-microparticle aggregate is compared to a corresponding calibration curve to find the concentration of each target compound in the fluid sample.

In some other embodiments of this aspect of the present invention, different sizes of functionalized microparticles are used with a single-channel or multi-channel resistive pulse sensor to find the concentration of two or more target compounds. In some of these embodiments, different sized functionalized microparticles are prepared. When these are added to the sample, the compound-microparticle aggregates formed for each target compound are also different sizes. A resistive pulse sensor, multichannel resistive pulse sensor, or particle counter or comparable device then simultaneously measures the number and size of the each compound-microparticle aggregate in the sample. Because the functionalized microparticles used for each target molecule are a different size, their corresponding compound-microparticle aggregates will also be different sizes and it is possible to determine which functionalized microparticles and/or compound-microparticle aggregates correspond to each one of the target compounds and to calculate volume and number fractions for each target molecule, as set forth above. These volume and number fractions can then be compared to corresponding calibration curves prepared as set forth above, to find the concentration for each target compound.

In some other embodiments of this aspect of the present invention, different colors of functionalized microparticles may be used to find the concentrations of two or more target compounds. In some of these embodiments, different colored functionalized microparticles are prepared. When these are added to the sample, the compound-microparticle aggregates formed for each target compound are also different colors. In these embodiments, an optical microscope or other comparable device capable of both counting the number and/or size of the functionalized microparticles and compound-microparticle aggregates, and recognizing the color of each. The number and/or size of the functionalized microparticles and/or compound-microparticle aggregates are then simultaneously recorded for each color. Because the functionalized microparticles used for each target molecule are a different color, their corresponding compound-microparticle aggregates will also be a different color, and it is possible to determine which functionalized microparticles and/or compound-microparticle aggregates correspond to each one of the target compounds and to calculate volume and number fractions for each target molecule, as set forth above. These volume and number fractions can then be compared to corresponding calibration curves prepared as set forth above, to find the concentration of each target compound in the sample.

In some other embodiments of this aspect of the present invention, functionalized microparticles having different fluorescence spectra may be used to find the concentrations of two or more target compounds. In some of these embodiments, functionalized microparticles having a different fluorescence spectrum are prepared for each of the two or more different target compounds and when these are mixed with the sample as set forth above, the compound-microparticle aggregates formed will have a different fluorescence spectrum for each of the compounds being measures.

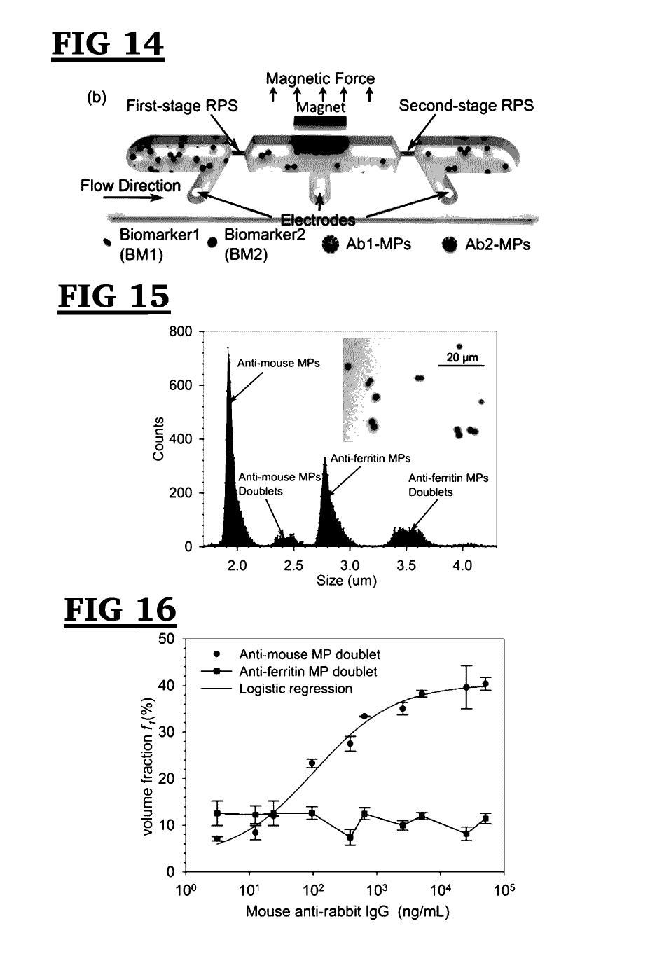

In some other embodiments of this aspect of the present invention, functionalized microparticles having different magnetic properties may be used with a single-channel or multi-channel resistive pulse sensor to find the concentration of two or more target compounds. In some of these embodiments, functionalized microparticles having different magnetic properties are prepared, as described above. When these are added to a sample, the compound-microparticle aggregates formed for each target compound also have different magnetic properties. An example resistive pulse sensor for use with functionalized microparticles having different magnetic properties is shown in FIG. 14. Because the functionalized microparticles used for each target molecule have different magnetic properties, their corresponding compound-microparticle aggregates will also be different sizes and it is possible to determine which functionalized microparticles and/or compound-microparticle aggregates correspond to each one of the target compounds and to calculate volume and number fractions for each target molecule, as set forth above. These volume and number fractions can then be compared to corresponding calibration curves prepared as set forth above, to find the concentration for each target compound.

In these embodiments, the number and size of the compound-microparticle aggregates for each of the target compounds may be measured by a fluorescent microscope or flow cytometer and the volume or number fraction of the compound-microparticle aggregates for each target compound calculated. In these embodiments, separate calibration curves are prepared for each for each target compound and its corresponding functionalized microparticle and the measured volume fraction or number fraction of each of compound-microparticle aggregate is compared to a corresponding calibration curve to find the concentration of each target compound in the fluid sample.

### EXPERIMENTAL

Human ferritin was used as a model biomarker to prove the concept of microparticle-based immunoaggregation for macromolecular biomarker detection. Ferritins are a class of iron storage proteins and they are widely distributed in vertebrates, invertebrates, plants, fungi and bacteria. For human, the increase of iron levels can promote the growth of a wide variety of tumor and infectious microorganisms. Ferritin measurement is considered the most reliable method for the evaluation of iron stores in serum. It was believed that the volume fraction of MPs aggregates to the total volume of particles was contingent on the antigen concentration at a given microparticle concentration. To test this, it was important to quantitatively correlate the biomarker concentration to aggregate size, counts and volume. First the immunoaggregation behaviour of ferritin and MP in the simple solution, PBS was measured. The ferritin concentrations ranged from 1.0 ng/ml to 208 ng/ml and the concentration of MP was kept constant at 53.40 μg/ml (3.5×10³ particle/μl). After aggregates were formed at each ferritin concentration, samples were loaded to a resistive pulse sensor, as described above, for aggregates analysis. For each test, 20 μl sample was measured at a flow rate of 80 μl/hr.

FIG. 3 shows typical relative pulse caused by particles. Each pulse represents one particle passing the sensing channel. Ferritin and MP concentrations were 41.6 ng/ml and 53.40 μg/ml. Single MPs, 2-MP aggregates and 3-MP aggregates were identified by the magnitude of the resistive pulses (δR/R), which is proportional to the particle volume (˜d³) according to Equation 1 above, where R is the resistance of the sensing channel, d is the diameter of the particle, D and L are the characteristic diameter and the length of the rectangular sensing channel, F is the correction factor. In this design, D was calculated to be 11.29 μm by D=(4·A/π)^{1/2}, where A was the cross-sectional area of the sensing channel. F was taken to be 1.0. After 15 μl sample passed the sensing channel, the particle size was back calculated using Equation 1. Counts of single MPs. 2-MP aggregates and 3-MP aggregates are shown in FIG. 4.

FIG. 4 shows the counts and size distributions of MP aggregates at a ferritin concentration of 41.60 ng/ml. The measured diameters of single MPs, 2-MP and 3-MP aggregates were 2.80±0.21 μm, 3.50±0.19 μm and 3.98±0.12 μm, which matched well with the nominal diameters and calculated equivalent diameters of single MPs, 2-MP aggregates and 3-MP aggregates (2.80 μm, 3.53 μm and 4.04 μm, respectively). Note that the ratio of aggregates formed by more than 3 MPs to all particles was less than 1%, which can hardly be seen in FIG. 4. Similar tests were conducted at various ferritin concentrations ranging from 1.04 ng/ml to 208.00 ng/ml; counts of single MPs and aggregates were obtained using the same procedure. For each test, the volume fraction of aggregates (f), defined as volume ratio of aggregates to all detected particles, was calculated. Note that the detection limit of the immunoaggregation method is mainly controlled by the nonspecific aggregation of MPs. The nonspecific aggregation of MPs in PBS without ferritin was tested and the volume ratio of non-specific MP aggregates was 0.051±0.006. After subtracting the volume ratio of nonspecific aggregates, volume fractions of ferritin-MP aggregates at different ferritin concentrations were plotted in FIG. 7 as a function of the ferritin concentration. To ensure repeatability, at each ferritin concentration, 10 samples were prepared and measured.

As shown in FIG. 7, the volume fraction of aggregates, f, increased with the increase of ferritin concentration in the range of 1.04 ng/ml to 62.40 ng/ml; the max volume fraction (35.4%) occurred at 62.40 ng/ml. Above 62.40 ng/ml, f reduced with the increase of the ferritin concentration. While not wishing to be bound by theory, it is believed that this is because excessive ferritins at higher concentrations possibly saturated anti-ferritin antibodies (Abs) on the surfaces of MPs; hence the number of unreacted anti-ferritin Ab on MP was too low to cause the aggregation.

Nonspecific binding of non-target biomolecules to capture probes or sensing surfaces has been a long standing challenging issue for nearly all biomarker detection in complex media, such as blood, urine, body fluid, etc. As set forth above, the nonspecific binding may cause the false positive result and decrease the detection sensitivity. To evaluate the performance of the aggregation-based resistive pulse sensor in complex media, 10% FBS was used as a complex media to mimic a real disease diagnosis condition. Different amount human ferritin was added to 10% FBS to different concentrations (0.1 ng/ml to 416 ng/ml). MP solutions (13.35 μg/ml, 53.40 μg/ml and 213.40 μg/ml) were mixed with human ferritin solution in 10% FBS for testing, and MP solutions were also mixed with 10% FBS without human ferritin separately as negative controls. Volume fractions of nonspecific aggregates for the negative control were 0.045±0.008, 0.054±0.011 and 0.049±0.002 for MP concentrations of 13.35 μg/ml, 53.4 μg/ml and 213.40 μg/ml, respectively, which are close to that in PBS at the concentration of 53.4 μg/ml. These results indicate that MPs are stable in the complex medium and nonspecific binding of non-target protein has no effect on the aggregation of MPs. FIG. 6 shows a clear relationship between the biomarker concentration and the volume fraction of aggregates. With the MP concentration of 53.4 μg/ml, the detection range was from 0.1 ng/ml to 62.40 ng/ml, which is close to that in PBS. With the MP concentration of 53.40 μg/ml (curve), the max volume fraction also occurs at 62.40 ng/ml (see FIG. 7), and f (31.1%) was comparable to that in PBS (35.4%). This result further confirms the aggregation-based biomarker detection method is insensitive to other biomolecules present in the complex media, implying that this approach can be applied to biomarker detection in complex media, i.e. human blood.

Furthermore, FIG. 6 shows that another significant advantage of the aggregation based biomarker detection methods of the present invention is that the detection range can be tuned by adjusting the concentration of MP. Shown as the black line with triangles, the detection range can be shifted to the lower concentration range (0.1 ng/ml to 10.4 ng/ml) by using a lower MP concentration (13.35 μg/ml)(black line with circles). Similarly, a higher detection concentration range (0.1 ng/ml to 208 ng/ml) was achieved with a higher MP concentration (213.40 μg/ml), as shown in the black line with squares. The normal range human ferritin is about 18 to 270 ng/ml. The detection range can be covered and is comparable with commercial available kits at sub ng/ml scale. As set forth above, although higher MP concentration provide a larger detection range, a lower MP concentration is more sensitive to lower biomarker concentration. Hence, for a biomarker concentration exceeding the upper detection range, the correct biomarker concentration can be determined by adding a test with increased microparticle concentration.

Results suggest that the label-free biomarker assay via aggregation offers high sensitivity, high selectivity and portability, and requires no complex setup and sample preparations. From the measured aggregates volume fraction, the biomarker concentration can be determined. Although ferritin-MP aggregation was not formed in the microchannel, it was possible to integrate an aggregation section and the resistive pulse sensor into one chip. In this testing, human ferritin as a real biomarker was detected in 10% FBS with a detection range from 0.1 ng/ml to 208 ng/ml. To further decrease the lower detectable biomarker concentration, one possible approach is to use the antibody with a higher binding affinity to the given biomarker, which is expected to improve the volume fraction at lower biomarker concentrations. Since the binding of antibodies to the antigens is a reversible and concentration-dependent process, the antibody with the higher binding affinity is able to capture the antigen and subsequently cause the aggregation at a lower concentration. Under flow conditions, a stronger interaction between the antigen and the antibody can reduce the disassociation of aggregates caused by the shear stress in the microfluidic channel and subsequently increase the detection sensitivity. Since lower detection limit is affected by nonspecific aggregation, using anti-fouling materials to avoid nonspecific aggregation of MPs is another promising method to extend lower detection range. Additionally, as mentioned above, by decreasing and increasing the concentrations of MPs, the lower and upper detection limits can be extended. Compared to conventional sandwich ELISA method that will take hours to finish, this approach only needs less than an hour. Further, a multiplexed multichannel resistive pulse sensor can be used to further reduce the detection time and detect multiple biomarkers in parallel. With these capabilities, the proposed immunoaggregation based label-free biomarker assay can potentially lead to lost cost, portable device to detect multiple biomarkers rapidly for clinical and biomedical research.

Additional experiments were also conducted to evaluate the viability the methods of the present invention using less complex and expensive counting techniques. In these experiments, goat IgG was used as a model biomarker and FITC-labeled rAb was conjugated to MP as a capture ligand/probe. The qualitative aggregation phenomenon of rAb-MP was studied using the fluorescent microscope though a GFP filter. Fluorescent microscope images (FIG. 11) show the dispersion state of particles in the rAb-MP solution of 53.4 μg/mL and the rAb-MP solution mixed with goat IgG with a final concentration was 36 ng/mL. In the absence of goat IgG, most rAb-MPs uniformly distributed and mostly separated from each other. After mixed with goat IgG, a large amount of rAb-MP aggregates formed. FIG. 13A-B shows the particle size distribution for rAb-MP solution with and without goat IgG at the same concentration with previous microscope study, which were measured by Accusizer. In both samples, multiple peaks were observed in the particle size frequency distribution curves generated by the Accusizer software, however individual particles (<3 μm) dominated (89%) in MP and Ab-MP samples. The small amount of larger particles (>3 μm) were formed by the nonspecific aggregation of individual particles. As shown in FIG. 13B, the percentage of larger particles ranging from 3 μm to 6 μm dramatically increased, which indicated the rAb-MP aggregates went up. The decrease of individual particles was caused by the aggregation triggered by the antigen. The result indicates that a large amount of rAb-MP aggregates can be formed because of the addition of biomarker.

Quantitative detection of biomarker is needed for many applications. As set forth above, at a given particle concentration, the number or volume ratio of aggregates to total particles is proportional to the target molecule concentration. To test this, the immunoaggregation behaviour of rAb-MP as a function of goat IgG concentration in PBS solution was measured by using the IX-81 microscope under the bright field mode. The concentration of rAb-MP was kept constant at 53.4 μg/mL and mixed with different concentrations of goat IgG ranging from 0.1 ng/mL to 320 ng/mL. Single rAb-MP and 2-rAb-MP aggregates, 3-rAb-MP aggregates and 4-rAb-MP aggregates for each goat IgG concentrations were recognized and counted separately through recorded microscope images. The aggregates formed by more than 4 rAb-MPs were neglected in the calculation, since they were less than 1%. To evaluate the nonspecific aggregates, the negative control sample of rAb-MP in PBS without goat IgG was also counted; the number fraction of nonspecific rAb-MP aggregates was 6.8±0.01%. The number fraction of rAb-MP aggregates for each goat IgG concentration was calculated by subtracting the nonspecific aggregates volume fraction value (6.8%) and had a clear relationship with goat IgG concentration as shown in FIG. 13A-B. At least 3 samples were prepared and counted for each goat IgG concentration. The max number fraction (fn=65.7%) of rAb-MP aggregates were achieved at 40 ng/mL of goat IgG. Within the range from 0.1 ng/mL to 40 ng/mL, the volume fraction of aggregates went up with the increase of goat IgG concentration, while it went down with the increase of goat IgG concentration above the turning point (40 ng/mL). It is believed that this is because goat IgG at high concentrations saturated rAb on MPs; therefore the number of unreacted rAb on MP was too low to aggregate.

Human ferritin was used as a real biomarker to validate whether MP-based immunoaggregation assay could be applied for the real human biomarker detection. Ferritins exist in many organisms, including vertebrates, invertebrates, plants, fungi, and bacteria, and they function as iron storage proteins. The abnormal level of ferritin in serum can be used as an indicator of various human disease, such as tumors and infectious microorganisms. Ferritin measurement is considered to be a reliable method for the evaluation of iron stores. Goat anti-human ferritin polyclonal antibody (gAb) were conjugated with MP to form gAb-MP and used as a capture probe.

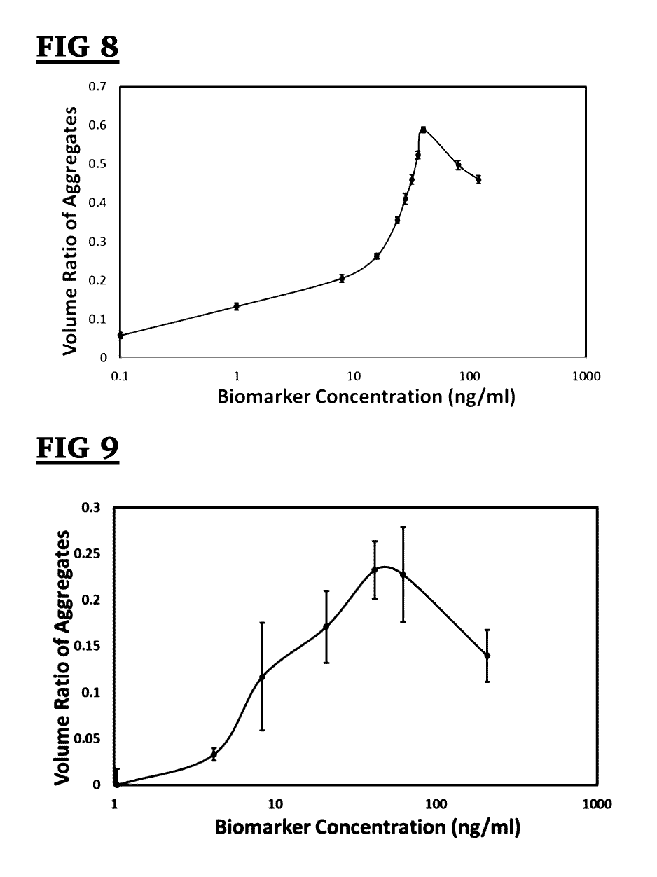

The samples with different concentrations of human ferritin antigen ranging from 1.04 ng/mL to 104 ng/mL in PBS and the constant gAb-MP concentration (53.4 μg/mL) were measured by the Accusizer. The volume fraction (fv) of gAb-MP aggregates (3.0 μm˜10 μm) to all particles (1.5 μm˜10 μm) were calculated and were plotted versus ferritin concentration (FIG. 8). Since the accusizer does not directly provide the information about how many smaller particles a larger particle is composed of, the volume fraction (fv) of larger particles to all particles more directly reflects the aggregation behavior of gAb-MP in this case. To ensure repeatability, at each ferritin concentration, 5 samples were prepared and measured. FIG. 8 shows that the same trend with goat IgG as the biomarker: The maximum volume fraction (fv=23.2%) of gAb-MP aggregates was achieved at the ferritin concentration of 41.6 ng/mL. Within the range from 1.04 ng/mL to 41.6 ng/mL, the volume fraction of gAb-MP aggregates to all the particles follows an upward trend with the increase of ferritin concentration. At higher ferritin concentrations (>41.6 ng/mL), the volume fraction of gAb-MP aggregates decreased with the increase of ferritin concentration.

As set forth above, detection of biomarkers is a challenge at low concentrations in complex media, such as body fluid, blood, urine, etc., since the nonspecific binding of biomolecules to the capture probes or sensing surfaces may cause the false positive result and decrease the detection sensitivity. To evaluate the feasibility of the aggregation assay for biomarkers in a complex medium, 10% FBS was used as the solution to replace PBS with 0.1% BSA to mimic a real detection environment. gAb-MP at two final concentrations, 53.4 μg/mL and 213.4 μg/mL, were mixed with human ferritin solution in 10% FBS. The solution with 10% FBS and the same concentration of gAb-MP but without ferritin was used as the negative control. Each sample was observed using the microscope under bright field mode; individual gAb-MPs, 2-gAb-MP aggregates, 3-gAb-MP aggregates and 4-gAb-MP aggregates were counted separately. More than 1000 particles were counted for each sample and 3 samples were counted for each ferritin concentration. The number fraction (fn) of nonspecific aggregates of the negative control were 6.8±0.3% and 6.8±0.1% for gAb-MP concentrations of 53.4 μg/mL and 213.4 μg/mL respectively. The low number fractions of the nonspecific gAb-MP aggregation suggest that gAb-MPs were stable in 10% FBS. The number fraction of aggregates of each sample was obtained by subtracting the number fraction value of the nonspecific aggregates. FIG. 9 shows that for the lower gAb-MP concentration (53.4 μg/mL), the detection range was from 0.1 ng/mL to 62.4 ng/mL. The result demonstrates that microparticle-based immunoaggregation assay for biomarker detection is insensitive to other biomolecules in the complex media, implying that this method can be applied for biomarker detection in complex media.

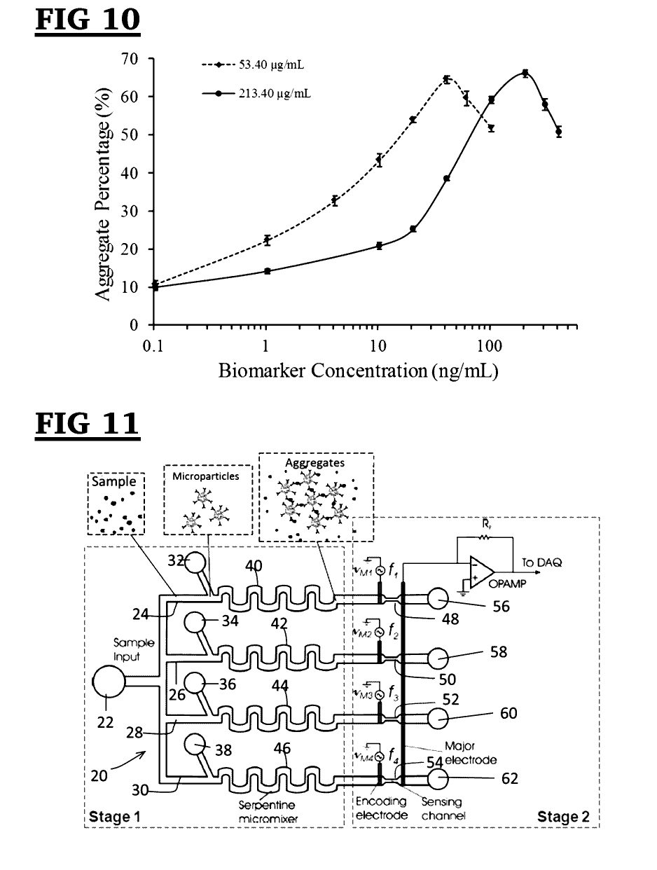

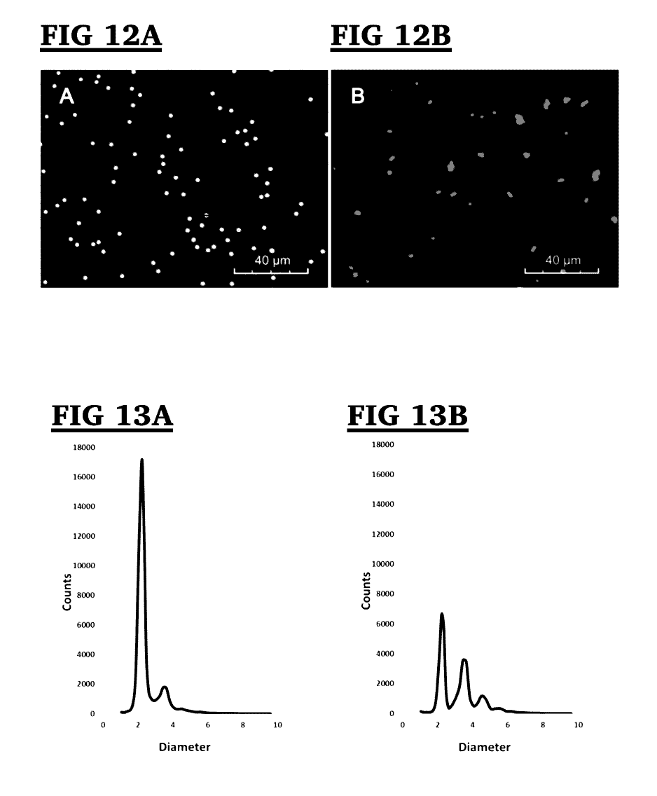

Since different biomarkers exist at different concentrations, it is highly desired that the detection range of the assay be adjustable to match the various concentrations of different biomarkers. Based on the aggregation principles described above, the detection range can be tuned by changing the Ab-MP concentration. FIG. 10 shows the detection range can be shifted to the higher concentration range (0.1 ng/mL to 208 ng/mL) by using a higher concentration of gAb-MP (213.4 μg/mL). It has been found that the detection range can be changed by adjusting the Ab-MP concentration for different biomarkers. Furthermore, two particle concentrations can be used to validate the accuracy of the result for samples with an unknown concentration range.

It is believed that this detection method will be particularly useful for hospitals or laboratories that need rapid clinical detection but lack immediate access to analytical instruments. Since the capture probe, Ab-MP conjugates could be prepared before the detection, the assay time for this method is less than 1 hour, which is shorter compared to the conventional ELISA method. Furthermore, the quantitative or qualitative detection of the biomarker can be conducted using optical microscopes with a hemocytometer, which are inexpensive and usually available in hospitals and biological labs. If an equal amount of particles are added to different samples with the same volume, the concentration of total particles in all samples will be the same. Since the volume of the fluid in the hemocytometer is fixed, the number of the aggregates in the hemocytometer can be directly linked to the antigen concentration. Therefore, it is only necessary to determine the number of aggregates, instead of recording both the counts of aggregates and individual particles. Moreover, the software for counting the particles and aggregates, such as Image J, could be used to replace manual counting to further reduce the assay time.

It is expected that complex samples, such as blood and body fluid, may cause higher non-specific aggregations that will increase the noise and detection limit. However, the detection limit of for the methods of these embodiments of the present invention is 0.1 ng/mL, which is lower than that of commercial available human ferritin ELISA kits (approximately 1 ng/mL), so the blood sample can be conveniently diluted to a suitable concentration before detection. On the other hand, as is demonstrated, the detection range can also be tuned by adjusting the Ab-MP concentration. For concentrated blood sample, a lower Ab-MP concentration can be used to achieve a lower detection range. The same method can be used to detect multiple biomarkers simultaneously, as set forth above. As described above, microparticles with different color and capture probes can be premixed and then added to the sample. Each type of the biomarker will cause the aggregation of microparticles with the specific color. If the sample contains different types of biomarkers, aggregates with different color can be detected. For the rapid qualitative detection, given the fixed sample volume and particle concentration, the threshold value of aggregates can be predetermined and sample can be considered as the positive once the number of aggregates is over the threshold value.

In another set of experiments, multiple types of antibody functionalized microparticle (Ab-MPs) functionalized by different antibodies with different size and magnetic properties were used for a multiplexed assay. The specific binding between one type of biomarkers and its specific Ab-MPs cause the formation of aggregates of that Ab-MPs. A two-stage micro resistive pulse sensor (RPS) was used to differentiate and count the Ab-Mps aggregates triggered by differentiate biomarkers in terms size and magnetic property for multiplexed detection. As set forth above, the volume fraction of the Ab-MPs aggregates indicates the concentration of the target biomarker. In these tests, human ferritin and mouse anti-rabbit IgG were used as target biomarkers to trigger the aggregation of two Ab-MPs, anti-ferritin Ab and anti-mouse IgG Ab functionalized MPs, in 10% fetal bovine serum (FBS), which was used to mimic a complex media. It was found that the volume fraction of Ab-MPs doublets increased with increased biomarkers concentrations. The detection ranges from 5.2 ng/ml to 208 ng/ml and 3.1 ng/ml to 51.2×103 ng/ml were achieved for human ferritin and mouse anti-rabbit IgG. This bioassay chip was able to quantitatively detect multiple biomarkers in a single test without any labeling process, and hence promises to become a useful tool for rapid detection of multiple biomarkers biomedical research and clinical applications.

Sample solutions containing biomarkers were mixed with Ab-MPs mixture to form immunoaggregates. Biomarker 1 (BM1), specific to Ab1, triggered the aggregation of antibody (Ab1) functionalized microparticles, Ab1-MPs. The volume fraction of Ab1-MPs doublets to all single Ab1-MPs probes and their doublets is indicative of the BM1 concentration. Similarly, BM2 in the sample induces the formation of Ab2-MPs. As shown in FIG. 14, biomarker sample and Ab1-MPs are mixed on the sensor chip. Due to the use of relatively large micro-sized beads for the immunoaggregation, the number of formed doublets was much higher than that of the formed triplets The formed doublets are detected by the 1st RPS, which can accurately measure the sizes and count the number of Ab-MPs and their aggregates. By selecting appropriate microparticles to ensure the Ab2-MPs doublet was smaller than a single Ab1-MP, the 1st RPS can differentiate Ab1-MP doublet, Ab2-MP doublet from single microparticles; hence the concentration of each biomarker can be measured by the 1st stage RPS from the volume fraction of the doublet induced by this biomarker.

Furthermore, it has been found that if MP1 conjugated with Ab1 are magnetic particles while MP2 conjugated with Ab2 are non-magnetic particles, Magnetic particles (Ab1-MPs) and their aggregates are captured by the capture chamber where an external magnetic field is applied. Hence only non-magnetic particles (Ab2-MPs) and their aggregates are detected by the 2nd RPS, while the 1st RPS detects all particles and aggregates. The difference of aggregates measured by the 1st RPS and the 2nd RPS are the magnetic particle aggregates, which are indicative of concentration of the Ab1. Hence with the two stage resistive pulse sensing device, multiple biomarkers can be detected in terms of size and magnetic property of the formed aggregates

Specifically, to prove the biomarker concentration measurement in terms of the size of formed doublets, the mixture of Ab-MPs probes were mixed with two model biomarkers, the mouse anti-rabbit IgG with a concentration of 24.0 ng/mL and human ferritin with a concentration of 208 ng/mL. The mixture of Ab-MPs probes consisted of 4.7×103 count/μL of anti-mouse MPs (2.0 μm in diameter) and 1.4×104 count/μL of anti-ferritin MPs (2.8 μm in diameter).

FIG. 15 shows the counts and size distributions of the two types of Ab-MPs and their aggregates. From left to right, the first two peaks centered at 2.00±0.06 μm and 2.50±0.08 μm represent the distribution of single and doublet of anti-mouse MPs. The measured diameters matched well with calculated equivalent diameters of anti-mouse MPs doublets, which are 2.52 μm. The third and fourth peaks centred at 2.88±0.08 μm and 3.61±0.11 μm represent the single and doublet of anti-ferritin MPs. The calculated equivalent diameters of anti-ferritin MPs doublet are 3.53 μm, which also matched with the measured result. The volume fraction of anti-mouse MPs doublets (f₁) was defined as volume of anti-mouse MPs doublets to all detected anti-mouse MPs and doublets. Similarly, the volume fraction of anti-ferritin MPs doublets was defined as the volume ratio of anti-ferritin MPs doublets to all detected anti-ferritin MPs and doublets. The result in FIG. 15 shows that the 1st-stage RPS was able to differentiate two types of Ab-MPs probes and their doublets according to their size distribution.

Next, to prove the volume fraction f₁ can be correlated to the concentrations of mouse anti-rabbit IgG, similar tests were conducted at various mouse anti-rabbit IgG concentrations ranging from 3.1 ng/mL to 51.2×103 ng/mL, while human ferritin concentration was kept a constant of 41.6 ng/mL. Note that the detection limit of the this aggregation method is mainly controlled by the non-specific aggregation of MPs26. The nonspecific aggregation of anti-mouse MPs in 10% FBS without mouse anti-rabbit IgG were tested and the volume ratio of non-specific anti-mouse MPs doublet were 17.9±0.4%. After subtracting the volume ratio of non-specific doublets, volume fractions of anti-mouse MPs doublets at different mouse anti-rabbit IgG concentrations were plotted in FIG. 16 as a function of the mouse anti-rabbit IgG concentrations. To ensure repeatability, at one mouse anti-rabbit IgG concentration, 5 aggregation samples were prepared and measured.

FIG. 16 shows the correlation between volume fraction of anti-mouse MPs doublets and anti-rabbit IgG ranging from 3.1 ng/mL to 51.2×103 ng/mL (circles), which can be fitted with a 4-parameter logistic function (solid curve):

$${f}_{1} ( x ) = 0.030 + \frac{0.37}{1 + {( x / 106.25 )}^{- 0.69}} \qquad ( Equation 6 )$$

The coefficient of determination (R₂) of the fitted curve was 0.9883. The volume fraction of anti-mouse-MPs doublet, f₁, was increased with the increase of mouse anti-rabbit IgG in the range of 3.1 ng/mL to 51.2×103 ng/mL. The max volume fraction (f₁=40.3%) occurred at 51.2×103 ng/mL. The average volume fraction of anti-ferritin MPs doublets was 11.1±2.0% (line with squares).

Note that the calculated equivalent average diameters of anti-mouse MPs triplets are 2.88 μm, which overlaps with the diameter range of anti-ferritin MPs and may cause a false count of anti-ferritin MPs. To evaluate the volume fraction of formed anti-mouse MPs triplets, control experiments were conducted using only mouse anti-rabbit IgG with a concentration from of 3.1 ng/ml to 51.2×103 ng/ml, and only one Ab-MPs, anti-mouse MPs with a concentration of 4.6×103 count/μl, the same as used in MPs mixture. From the RPS measurement, the triplet concentration was ranged from 154 to 500 counts/μL. Hence if ferritin and anti-ferritin MPs are added to the solution with a concentration of 1.4×104 counts/μl to from aggregates, the volume fraction of anti-mouse MPs triplets to total volume of anti-ferritin MPs and aggregates were estimated to range from 1.5% to 3.8% at all anti-rabbit IgG concentrations. The value was 1.5% a anti-rabbit IgG concentration of 24 ng/ml. Hence it was determined that the error caused by the size overlapping of anti-mouse MPs triplets and anti-ferritin Abs can be ignored.

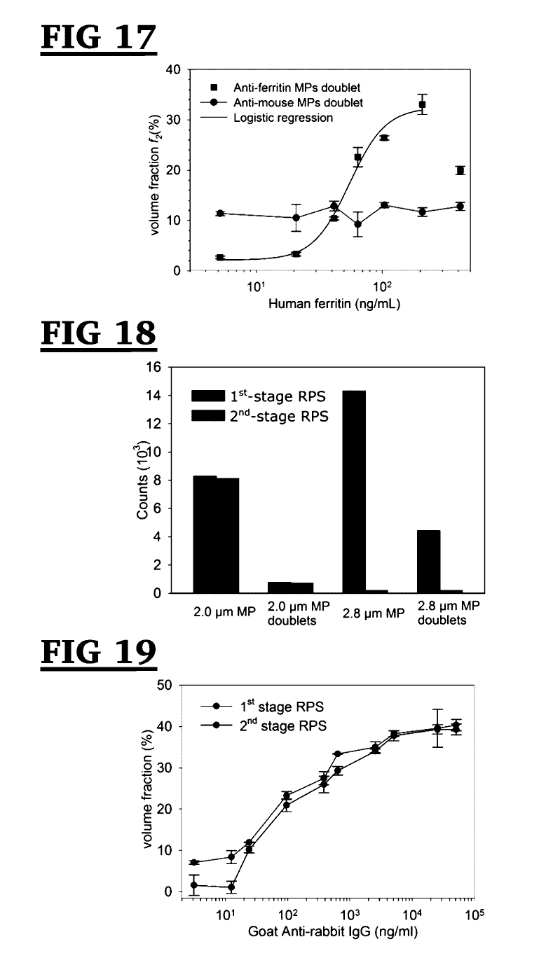

Next, it was determined that the volume fraction of anti-ferritin MPs doublets, f₂, was correlated to the concentrations of human ferritin. In this experiment, the concentration of human ferritin mouse anti-rabbit IgG was varied from 5.2 ng/mL to 208 ng/mL; while mouse anti-rabbit IgG concentration was kept a constant of 24.0 ng/mL, as shown in FIG. 17.

$${f}_{2} ( x ) = 0.02 + \frac{0.30}{1 + {( x / 55.00 )}^{- 3.02}} \qquad ( Equation 7 )$$

The correlation between volume fraction of anti-ferritin MPs doublets and human ferritin ranging from 5.2 to 208 ng/mL was also fitted with a 4-parameter logistic curve (FIG. 17, solid curve). The coefficient of determination (R₂) of the fitted curve was 0.9870. The volume fraction of anti-ferritin MPs aggregates was increased from 2.7% to 33.1% with the increase of human ferritin in the range of 5.2 ng/mL to 208 ng/mL. With a fixed IgG concentration of 24 ng/ml, the volume fraction error caused by the anti-mouse MPs triplets was estimated to be approximately 1.5%. Hence using the multiplexed aggregation assay we can safely measure the ferritin concentration as low as 5.2 ng/ml. The max volume fraction (33.1%) occurred at 208 ng/mL. above 208 ng/mL, the volume fraction f₂ was reduced with the increase of ferritin concentration. It is believed that this is because the high concentration of ferritin would saturate anti-ferritin Ab on the surfaces of Ab-MP; hence the number of unreacted anti-ferritin Ab on anti-ferritin MP was too low to cause the aggregation.

The measured average volume fraction of anti-mouse MPs doublets was 11.6±1.4% for the constant mouse anti-rabbit IgG concentration of 24 ng/mL. Using Equation 6 and anti-rabbit IgG concentration of 24 ng/mL, the calculated volume fraction of anti-mouse MPs doublets was 12.8%, which matched with the measurement value (11.6±1.4%) well. Using Equation 7 and the human ferritin concentration of 41.6 ng/mL, the calculated volume fraction of anti-ferritin MPs was 11.3%, which also matched well with measurement result in FIG. 16 (11.1±2.0%). The results shown in FIGS. 16 and 17 clearly demonstrate 1) the volume fraction of Ab-MPs doublets formed by two Ab-MPs probes, anti-mouse MPs and anti-ferritin MPs, are correlated to the concentrations of rabbit anti-mouse IgG and human ferritin in a mixture; and 2) the 1st-stage RPS was capable to differentiate two types of Ab-MPs probes and their doublets according to their size distribution.

Next, experiments were conducted to prove the two biomarkers can also be detected in terms of the magnetic property of the aggregates. The magnetic MPs aggregates are correlated to the anti-ferritin Ab, and the non-magnetic MPs aggregates are correlated to the mouse anti-rabbit IgG. An external magnet was used to capture magnetic particles in the capture chamber while the non-magnetic particles were counted by the 2nd-stage RPS.

In order to prove that the external magnet was able to capture magnetic MP with high efficiency, we compared the count and size distribution measured by the 1st and 2nd RPSs in following experiment condition: 208 ng/ml of ferritin, without mouse anti-rabbit IgG. The mixture of Ab-MPs probes were the same as used in the previous experiments. Counts of the different-sized particles are shown in FIG. 18. The capture efficiency was calculated as a ratio of the difference of magnetic particle (magnetic MP and its aggregates) counts between the 1st RPS and the 2nd RPS over all magnetic counts measured by the 1st RPS. From the measurements shown in FIG. 18, the capture efficiency was calculated to be greater than 98.0%, which is high enough to ensure accurate count of magnetic particles.

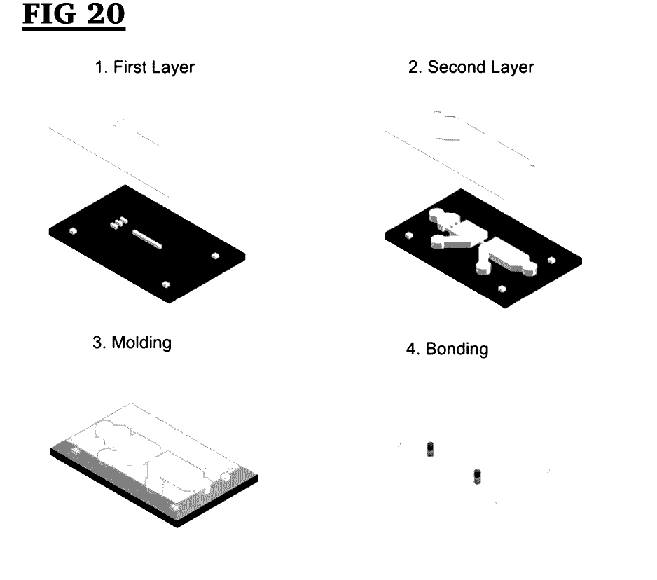

Next, to prove that 2nd-stage RPS can accurately count and size the non-magnetic MPs, we compared the measured volume ratio of non-magnetic doublets measured by the 1st-stage and 2nd-stage of RPS when mouse anti-rabbit IgG concentration was varied from 3.1 ng/mL to 51.2×103 ng/mL. As can be seen from the result shown in FIG. 19, the volume fraction ratio measured by the 1st RPS and 2nd RPS matches reasonably well when the IgG concentration ranges from 24.0 to 51.2×103 ng/mL; the measurement error ranges from 0.2% to 4.1%, which, it is believed, could be caused by disassociation of aggregates caused by the shear stress in the 1st RPS.

### EXAMPLES

The following examples are offered to more fully illustrate the invention, but are not to be construed as limiting the scope thereof. Further, while some of examples may include conclusions about the way the invention may function, the inventor do not intend to be bound by those conclusions, but put them forth only as possible explanations. Moreover, unless noted by use of past tense, presentation of an example does not imply that an experiment or procedure was, or was not, conducted, or that results were, or were not actually obtained. Efforts have been made to ensure accuracy with respect to numbers used (e.g., amounts, temperature), but some experimental errors and deviations may be present. Unless indicated otherwise, parts are parts by weight, molecular weight is weight average molecular weight, temperature is in degrees Centigrade, and pressure is at or near atmospheric.

### Example 1

### Resistive Pulse Sensor Fabrication and Testing Procedure

The resistive pulse sensor was fabricated using the standard soft lithography method. It consists of 1) a sensing channel with a width of 10 μm and a length of 30 μm to detect aggregates; 2) an on-chip filter with a pore width of 10 μm; and 3) a pair of Ag/AgCl electrodes to measure the resistive pulses. A two-layer SU8 mold, consisting of patterns for the sensing channel and the filter (with a thickness of 10 μm) and patterns for reservoirs (with a thickness of 40 μm), was created by a two-step photolithography process (See, FIG. 20). Microchannel, filters and reservoirs were formed by pouring polydimethylsiloxane (PDMS) onto the two-layer SU8 mold followed by degassing and curing. The filter blocks background particles larger than the sensing channel to ensure a continuous detection. This two-layer structure offers a higher sensitivity of the sensing channel without increasing the flow resistance of reservoirs.

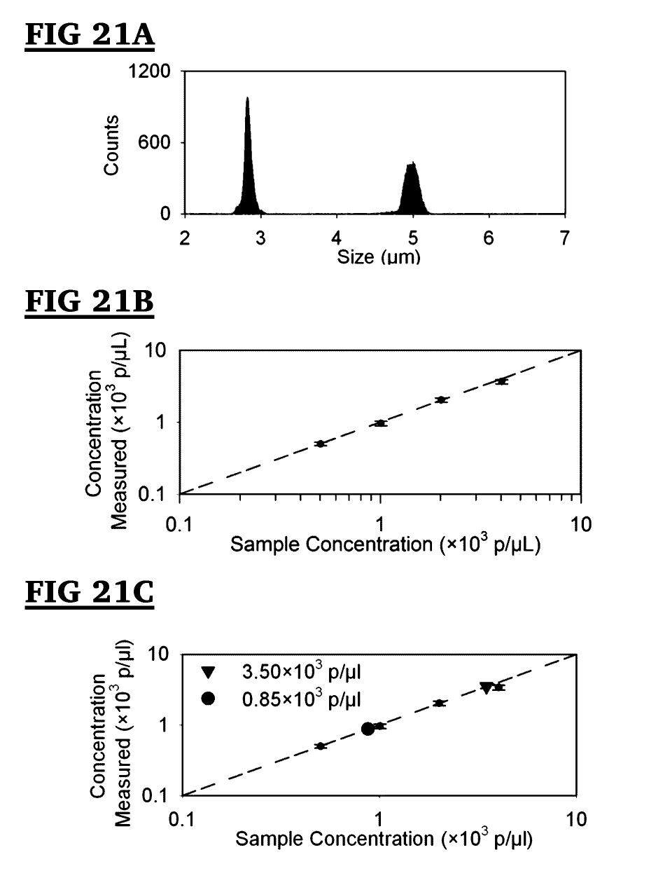

Next, PDMS microchannel was bonded onto a glass substrate after an oxygen plasma treatment (200 mTorr, 50 W, 50 s). A pair of Ag/AgCl electrodes (1 mm in diameter) was inserted on each side of the sensing channel via two punched 1-mm holes (see FIG. 20) to finish the fabrication of the resistive pulse sensor. The resistive pulse sensor was then tested using MPs with two standard sizes to ensure it could accurately measure the sizes and counts of MPs (See, FIG. 21A-C). Microparticles (MP) having diameters of MPs are 2.80 μm (Dynabeads M-280, Life Technologies, USA) and 5.00 μm (79633 FLUKA, Sigma Aldrich) were used to calibrate the counting and sizing performances of the device. The measured sizes were 2.80±0.16 μm and 4.91±0.37 μm. For concentration calibration, four MP concentrations of 500 p/μl, 1000 p/μl, 2000 p/μl and 4000 p/μl were used, as shown in FIG. 21A. The measured concentrations were 497±28 p/μl, 960±70 p/μl, 2025±138 p/μl and 3657±240 p/μl. Three sets of MP concentrations were used: 13.35 μg/ml (875 p/μl), 53.40 μg/ml (3500 p/μl), and 213.40 μg/ml (14000 p/μl). As show in FIG. 21B, the circle and triangle represent the particle concentration of 875 p/μl (13.35 μg/ml) and 3500 p/μl (53.40 μg/ml). The MPs with concentration of 213.40 μg/ml was diluted 1/4 and measured. For each test, 20 μl of sample was loaded into the inlet reservoir at a flow rate of 80 μl/hr driven by a syringe pump (KDS Legato 270, KD Scientific).

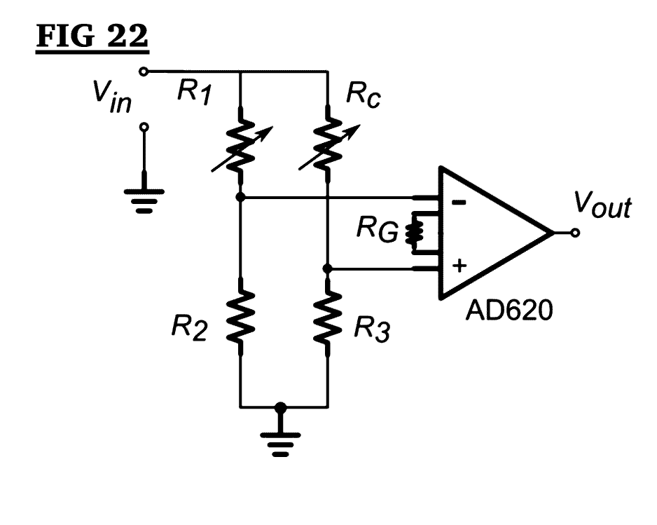

As can be seen from FIG. 22, the micro resistive pulse sensor was modeled as a variable resistor R_{c}; Resistors R₂=R₃=500 kΩ; A variable resistor R₁ ranging from 500 kΩ to 1 MΩ was set equal to match R_{c}. The gain of the difference amplifier, AD620, was programmed to be 50 using an external gain resistor R_{G}=1 kΩ. The input single Vᵢₙ=2.4 V was provide by a function generator (33220A, Agilent). The output signal was record by a data acquisition card (NI USB-6251, National Instruments) at sampling rate of 1 MHz. Finally, a custom peak detection algorithm (Matlab, MathWorks) was used to count the resistive pulse number and back calculate the particle size.

### Example 2

### Sample Preparation

To prepare antibody-functionalized MPs, firstly, streptavidin functionalized magnetic MPs with an average diameter of 2.80 μm (dynabeads M280, Life Technologies, USA) were diluted 1/65 in phosphate buffer saline (PBS, pH 7.4, Sigma-Aldrich, USA) containing 0.1% bovine serum albumin (BSA, Sigma-Aldrich, USA). The biotinylated goat anti-human ferritin antibody (anti-ferritin Ab, 6.5 mg/ml, US Biological, USA) was diluted 1/780 in PBS with 0.1% BSA. Next, 166.7 μl of diluted M280 MP solution was mixed with 166.7 μl diluted anti-ferritin Ab solution for 30 min in a thermal mixer at a speed of 650 rpm at room temperature. M280 MPs conjugated with biotinylated anti-ferritin Ab through the streptavidin-biotin binding. It has been found that further increasing the incubation time has little effect on the volume fraction of aggregates. The MP solution was then placed on a magnet to separate MPs from the solution; next the supernatant containing unconjugated anti-ferritin Ab was removed. MPs were then resuspended in PBS with 0.1% BSA to three concentrations, 13.35 μg/ml, 53.40 μg/ml and 213.40 μg/ml.

The biomarker solutions, human ferritin (US Biological, USA), with various concentrations ranging from 0.1 ng/ml to 416 ng/ml were prepared by serial dilution with PBS (0.1% BSA). 333.4 μl of MP solution was mixed with 166.7 μl of ferritin solution at different concentrations for 30 min in a thermal mixer at the speed of 650 rpm at room temperature. Ferritins caused the specific aggregation of MPs, which was detected by the resistive pulse sensor. Also in a parallel study, 10% fetal bovine serum (FBS, Sigma-Aldrich, USA) was used to dilute the human ferritin to different concentrations ranging from 0.1 ng/ml to 416 ng/ml using similar gradient dilution to mimic the real biomarker detection environment in complex media.

### Example 3

### Materials and Methods for Analysis of Sample

Streptavidin-functionalized Microparticle (MP) (Dynabeads M-280 with a diameter of 2.8 μm), biotinylated polyclonal rabbit anti-goat IgG (rAb) antibodies and goat anti-rabbit IgG (goat IgG) antibodies (labeled with Alexa Fluor 488) were bought from Life Technologies (Carlsbad, Calif., USA). Goat anti-human ferritin polyclonal antibody (gAb) and human ferritin were purchased from United States Biological (Salem, Mass., USA). NHS-Fluorescein, NHS-PEG4-Biotinyltion and Zeba spin desalting column were purchased from Thermo Scientific (Waltham, Mass., USA). Dimethyl sulfoxide (HPLC grade) was bought from Alfa Aesar (USA). Phosphate buffer saline (PBS, pH 7.4), and bovine serum albumin (BSA) were obtained from Sigma-Aldrich (St Louis, Mo., USA).

To prepare the immunoaggregation sample, MP and biotinylated rAb were diluted to 0.16 mg/mL and 6.4 ng/mL separately in PBS containing 0.1% BSA. Equal volumes of 166.7 μL of diluted MP solution and 166.7 μL of diluted rAb solution were mixed for 30 minutes on a thermo mixer agitated at 650 rpm at room temperature. Biotinylated rabbit anti-goat Abs were conjugated to MP to form rAb-MP through the streptavidin-biotin binding. The conjugated solution was placed on a magnet to separate rAb-MPs from the solution and the unconjugated Ab supernatant was discarded. The rAb-MPs were resuspended with PBS with 0.1% BSA to the concentrations of 53.4 μg/mL. Different concentrations of goat IgG, which was used as model biomarker, were prepared with a range from 0.1 ng/mL to 320 ng/mL. 333.4 μL of Ab-MP solution was mixed with 166.7 μL of goat IgG solutions at different concentrations for 30 min on a thermal mixer at 650 rpm at room temperature. The same procedure was used for human ferritin detection. Goat anti-human ferritin polyclonal antibody (gAb) functionalized MP (gAb-MP) were suspended in PBS with 0.1% BSA to two different concentrations, 53.40 μg/mL and 213.4 μg/mL. The concentration of human ferritin ranged from 0.1 ng/mL to 416 ng/mL in PBS with 0.1% BSA. In a parallel study, 10% fetal bovine serum (FBS, Sigma-Aldrich, USA) was used to dilute the human ferritin to different concentrations ranging from 0.1 ng/mL to 416 ng/mL to mimic the biomarker detection in the complex medium.

### Example 4

### Optical Methods of Counting and Sizing Particles after Immunoaggregation

To characterize the immunoaggregation, rAb-MP solutions with and without biomarker (goat IgG) were imaged with a fluorescent microscope (IX-81, Olympus, Japan) under a 40× objective lens though bright field filter and GFP filter (494/518 nm) respectively and analyzed with MetaMorph microscopy automation & image analysis software (Molecular Devices, CA, USA). The rAb-MP aggregates solutions formed under different goat IgG concentrations were dropped to a glass slide and covered with a cover slip. The number of rAb-MP, 2-rAb-MP aggregates, 3-rAb-MP aggregates and 4-rAb-MP aggregates were counted separately through the Olympus IX-81 fluorescent microscope under the bright field mode. To ensure accuracy and repeatability, more than 1000 particles were counted for each sample and 3 samples were prepared and tested for each goat IgG concentration. The number fraction of aggregates (fn) was defined as the ratio of the number of individual particles in aggregates to the number of all individual particles. The same characterization methods were used for the human ferritin detection.

To confirm the result, Ab-MP aggregates were diluted to 100 mL PBS with 0.1 mg/mL BSA and the size and counts of the particles in the sample were measured using a particle counter with a detection range of 0.5˜500 μm (Accusizer 780, PALS-Particle Sizing Systems, FL, USA). The volume fraction of aggregates (fv) was calculated as volume ratio of large particles (3.0 μm˜10 μm) to all particles (1.5 μm˜10 μm).

### Example 5

### Device Fabrication and Testing Procedure for Two Stage Resistive Pulse Sensor

The resistive pulse sensor was fabricated using the standard soft lithography method. It consists of 1) two sensing channels with a width of 10 μm and a length of 30 μm to detect aggregates, 2) a capture chamber with width of 1 mm and a length of 15 mm to capture magnetic aggregates and particles, 3) a pair of inlet and outlet reservoirs, and 4) three Ag/AgCl electrodes to measure the resistive pulses. A two-layer SU8 mold, consisting of patterns for the sensing channel (with a thickness of 10 μm), capture chamber and reservoirs (with a thickness of 40 μm), was created by a two-step photolithography. Microchannels, capture chamber and reservoirs were formed by pouring polydimethylsiloxane (PDMS) onto the two-layer SU8 mold followed by degassing and curing. This two-layer structure offers a higher sensitivity of the sensing channel without increasing the flow resistance of reservoirs. Next, PDMS microchannel was bonded onto a glass substrate after an oxygen plasma treatment (200 mTorr, 50 W, 50 s). Three Ag/AgCl electrodes (1 mm in diameter) was inserted on each side of the sensing channel via two punched 1-mm holes to finish the fabrication of the resistive pulse sensor. For each test, 50 μL of sample and 50 μL of Ab-MPs were mixed on the sensor chip for 30 mins, then driven through the two-stage RPS at a flow rate of 80 μL/hr by a syringe pump (KDS Legato 270, KD Scientific). An external magnet (Grade N42, 3.2 mm×3.2 mm×3.2 mm, K&J Magnetics, Inc.) was used to capture magnetic MPs in the capture chamber. Resistive pulse responses were record by a data acquisition card (NI USB-6251, National Instruments) at a sampling rate of 500 kHz (See measurement circuit in ESI). Note that the external magnet was placed 10 mm away from the 1^{st} stage RPS, as shown in FIG. 14. Using such a large distance reduces the possibility of magnetizing the magnetic microparticles before they enter the 1^{st} RPS; magnetized microparticles tend to form nonspecific aggregates, which will be counted as immuno aggregates and lead to errors in biomarker concentration measurement.

### Example 6

### Sample Preparation for Microparticles Having Magnetic Properties

To prepare a mixture of microparticles probes with different antibody functionalization, sizes and magnetic properties, two types of Ab-MPs were prepared separately first. Streptavidin functionalized polystyrene microparticles with an average diameter of 2.0 μm (Polybead Microspheres, Polysciences, Inc, U.S.A) was diluted in 1/20 in phosphate-buffered saline (PBS, pH 7.4, Sigma-Aldrich, U.S.A) containing 0.1% bovine serum albumin (BSA, Sigma-Aldrich, U.S.A). The biotinylated rabbit anti-mouse IgG (H+L) (Anti-mouse Ab, 1 mg/mL, Life Technologies, U.S.A) was diluted 1/180 in PBS with 0.1% BSA. Next 166.7 μL of diluted PS MP solution was mixed with 166.7 μL of diluted anti mouse Ab solution for 30 minutes in a thermal mixer at a speed of 650 rpm at room temperature. PS MPs conjugated with biotinylated anti mouse Ab through the streptavidin-biotin binding. The Ab-MP solution was then centrifuged at 10000 rpm to separate Ab-MPs from the solution and the supernatant containing unconjugated anti mouse Ab was removed for three times. MPs were then resuspended in PBS was 0.1% BSA to concentration of 142.3 μg/mL. Another type of Ab-MP was goat antihuman ferritin Ab (anti ferritin Ab, 6.5 mg/mL, US Biological, U.S.A) functionalized magnetic MP with an average diameter of 2.80 μm (Dynabeads M280, Life Technologies, U.S.A) and the preparation procedure was reported previously25. Two types of Ab-MPs were mixed together with equivalent volumes (166.7 μL each); the mixed solution was used as a mixture Ab-MPs probe.

Two biomarker solutions, human ferritin (US Biological, U.S.A.), with concentrations ranging from 5.2 to 416.0 ng/mL, and mouse anti-rabbit IgG (Life technologies, U.S.A) with concentrations ranging from 3.1 ng/mL to 51.2×10³ ng/mL were prepared by serial dilution with 10% fetal bovine serum (FBS, Sigma-Aldrich, U.S.A.). Two types of mixed biomarkers solution were prepared as follows: 1) the mouse anti-rabbit IgG ranging from 3.1 ng/mL to 51.2×10³ ng/mL while human ferritin were kept a constant concentration of 41.6 ng/mL; 2) human ferritin ranging from 2.6 ng/mL to 416.0 ng/mL while mouse anti-rabbit IgG was kept constant as 24.0 ng/mL. An amount of 333.4 μL of Ab-MPs mixture solution was mixed with 166.7 μL of the two biomarkers solutions mentioned above separately for 30 min in a thermal mixer at the speed of 650 rpm at room temperature. The biomarker causes specific aggregation of Ab-MPs, which was detected by the two-stage resistive pulse sensor.

In light of the foregoing, it should be appreciated that the present invention significantly advances the art by providing a method of biomarker detection and quantification that is a significant improvement over methods currently known in the art. While particular embodiments of the invention have been disclosed in detail herein, it should be appreciated that the invention is not limited thereto or thereby inasmuch as variations on the invention herein will be readily appreciated by those of ordinary skill in the art. The scope of the invention shall be appreciated from the claims that follow.

What is claimed is:

1. A method for measuring the concentration of a compound in a fluid comprising:
A. preparing a fluid sample containing an unknown concentration of a compound to be measured;
B. preparing a plurality of functionalized microparticles, said plurality of functionalized microparticles being functionalized to specifically interact with said compound;
C. combing said plurality of functionalized microparticles with said fluid sample containing an unknown concentration of said compound, wherein the interaction between said functionalized microparticles and said compound is sufficient to cause said functionalized microparticles to aggregate around the compounds in said sample, thereby forming compound-microparticle aggregates;
D. counting the number and size of said compound-microparticle aggregates;
E. calculating a volume fraction or number fraction of said compound-microparticle aggregates in said sample, based upon the number and size of said compound-microparticle aggregates in said sample;
F. preparing a calibration curve comprising the volume fraction or number fraction of compound-microparticle aggregates formed at known concentrations of said compound and said functionalized microparticles; and
G. comparing the volume fraction of the compound-microparticle aggregates found in said counting and calculating step to said calibration curve to find the concentration of said compound in said fluid sample.

2. The method of claim 1, wherein said plurality of functionalized microparticles have a diameter of from 0.5 μm or more to 10 μm or less.

3. The method of claim 1, wherein said plurality of functionalized microparticles have a diameter of from 0.2 μm or more to 0.5 μm or less.

4. The method of claim 1, wherein said plurality of functionalized microparticles have a diameter of from 0.5 μm or more to 5 μm or less.

5. The method of claim 1, wherein said plurality of functionalized microparticles have a diameter of from 5 μm or more to 10 μm or less.

6. The method of claim 1, wherein said plurality of functionalized microparticles have a diameter of from 10 μm or more to 50 μm or less.

7. The method of claim 1, wherein said plurality of functionalized microparticles are magnetic.

8. The method of claim 1, wherein said plurality of functionalized microparticles are functionalized with one or more capture ligands selected from the group comprising antibodies, proteins, peptides, nucleic acids, aptamers, poly/oligo/mono saccharides, and combinations thereof.

9. The method of claim 1, wherein said plurality of functionalized microparticles comprise polystyrene, latex, gold, silica, organic materials, inorganic materials or combinations thereof.

10. The method of claim 1, wherein said compound-microparticle aggregates comprise one compound to be measured and at least two functionalized microparticles.

11. The method of claim 1, wherein said compound to be measured is selected from the group consisting of ferritin, alanine transaminase (ALT), aspartate transaminase (AST), anti-hCG antibody, carcinoembryonic antigen (CEA), Alpha-Fetoprotein (AFP), AFP-L3, prostate specific antigen (PSA), C-reactive protein (CRP), estrogen receptor/progesteron receptor, receptor tyrosine-protein kinase erbB-2, (HER-2/neu), the epidermal growth factor receptor (EGFR), V-Ki-ras2 Kirsten rat sarcoma viral oncogene homolog (KRAS), UDP glucuronosyltransferase 1 family (UGT1A1), receptor tyrosine kinase (c-KIT), CD20 Antigen, CD30, fip1-like-1 fused with platelet derived growth factor receptor alpha (FIP1L1-PDGRFalpha), Platelet-derived growth factor receptors (PDGFR), Philadelphia Chromosome (BCR/ABL), PML/RAR alpha, thiopurine S-methyltransferase (TPMT), anaplastic lymphoma kinase (ALK), V-Ki-ras2 Kirsten rat sarcoma viral oncogene homolog (KRAS), serine/threonine-protein kinase B-Raf (BRAF), peptides, poly/oligo-saccharide, nucleic acids, lipoproteins, other biomolecules, virus, microplasma, bacteria, and combinations thereof.

12. The method of claim 1, wherein the concentration of said compound to be measured in said sample is from about 1 pg/mL or more to about 100 mg/mL or less.

13. The method of claim 1, wherein said functionalized microparticles further comprise a fluorescent molecule.

14. The method of claim 1, wherein the step of counting the number and size of said compound-microparticle aggregates performed using a resistive pulse sensor, an optical microscope, a fluorescence microscope, a flow cytometer or a particle counter.

15. The method of claim 14, wherein the step of counting the number and size of said compound-microparticle aggregates is performed using a resistive pulse sensor.

16. The method of claim 15, wherein the resistive pulse sensor further comprises a channel having an area, said plurality of functionalized microparticles has a projected area, and the projected area of one of said plurality of microparticles is from about 5% or more to about 50% or less of the area of said channel.

17. The method of claim 15, wherein said resistive pulse sensor has two or more channels for counting the number and size of said compound-microparticle aggregates.

18. The method of claim 1 wherein said fluid sample contains an unknown concentration of two or more different compounds to be measured;
the step of preparing a plurality of functionalized microparticles further comprises preparing a plurality of functionalized microparticles for each one of said two or more different compounds to be measured;
the step of combining further comprises forming a compound-microparticle aggregates for each of the compounds being measured;
the step of counting further comprises placing the sample containing compound-microparticle aggregates for each of the compounds being measured in a multichannel resistive pulse sensor, said multichannel resistive pulse sensor simultaneously measuring the number and size of the compound-microparticle aggregates for each of the compounds to be measured;
the step of calculating further comprising the step of calculating the volume fraction or number fraction of the compound-microparticle aggregates for each compound to be measured in said sample;
the step of preparing a calibration curve further comprises preparing a calibration curve for each for each compound to be measured in said fluid sample; and
the step of comparing further comprises comparing the volume fraction of each of compound-microparticle aggregates found in said counting and calculating steps to its corresponding calibration curve to find the concentration of each compound to be measured in said fluid sample.

19. The method of claim 1 wherein said fluid sample contains an unknown concentration of two or more different compounds to be measured;
the step of preparing a plurality of functionalized microparticles further comprises preparing functionalized microparticles of a different size for each one of said two or more different compounds to be measured;
the step of combining further comprises forming a compounds-microparticle aggregate for each of the compounds being measured;
the step of counting further comprises placing the sample containing compound-microparticle aggregates for each of the compounds being measured in a resistive pulse sensor, multichannel resistive pulse sensor, or particle counter, said resistive pulse sensor, multichannel resistive pulse sensor, or particle counter simultaneously measuring the number and size of the compound-microparticle aggregates for each of the compounds to be measured;
the step of calculating further comprising the step of calculating the volume fraction or number fraction of the compound-microparticle aggregates for each compound to be measured in said sample;
the step of preparing a calibration curve further comprises preparing a calibration curve for each for each compound to be measured in said sample; and
the step of comparing further comprises comparing the volume fraction or number fraction of each of compound-microparticle aggregates found in said counting and calculating steps to its corresponding calibration curve to find the concentration of each compound to be measured in said fluid sample.

20. The method of claim 1 wherein said fluid sample contains an unknown concentration of two or more different compounds to be measured;
the step of preparing a plurality of microparticles further comprises preparing a plurality of functionalized microparticles for each of said two or more different compounds to be measured, said functionalized microparticles for each of said two or more different compounds to be measured having a different color;
the step of combining further comprises forming a compounds-microparticle aggregate for each of the compounds being measured;
the step of counting further comprising placing the sample containing the compound-microparticle aggregates for each of the compounds being measured in an optical microscope and measuring the number and of the compound-microparticle aggregates for each of said colors;
the step of calculating further comprising the step of calculating the number fraction of the compound-microparticle aggregates present for each compound to be measured in said sample; the step of preparing a calibration curve further comprises preparing a calibration curve for each compound to be measured in said sample; and
the step of comparing further comprises comparing the number fraction of each of compound-microparticle aggregates found in said calculating step to its corresponding calibration curve to find the concentration of each compound to be measured in said fluid sample.

21. The method of claim 1 wherein said fluid sample contains an unknown concentration of two or more different compounds to be measured;
the step of preparing a plurality of functionalized microparticles further comprises preparing a plurality functionalized microparticles for each of said two or more different compounds to be measured, wherein the functionalized microparticles for each of said two or more different compounds to be measured have a different fluorescence spectrum;
the step of combining further comprises forming a compound-microparticle aggregates for each of the compounds being measured;
the step of counting further comprises placing the sample containing the compound-microparticle aggregates for each of the compounds being measured in a fluorescent microscope or flow cytometer, said optical microscope measuring the number of the compound-microparticle aggregates and said flow cytometer measuring the number and size of the compound-microparticle aggregates at the fluorescence spectrum for each one of the compounds to be measured;
the step of calculating further comprising the step of calculating the volume or number fraction of the compound-microparticle aggregates for each compound to be measured in said sample;
the step of preparing a calibration curve further comprises preparing a calibration curve for each for each compound to be measured in said sample; and
the step of comparing further comprises comparing the volume or number fraction of each of the compound-microparticle aggregates found in said counting and calculating steps to its corresponding reference curve to find the concentration of each compound to be measured in said fluid sample.
